(19) United States
(12) Patent Application Publication
(10) Pub. No.: US 2009/0324585 A1
(43) Pub. Date: Dec. 31, 2009

(54) COMPLEMENT INHIBITORY AGENTS AS THERAPEUTICS IN POSTTRAUMATIC AND DEGENERATIVE ARTHRITIS

(75) Inventors: William H. Robinson, Palo Alto, CA (US); V. Michael Holers, Denver, CO (US); Andrew L. Rozelle, Menlo Park, CA (US)

Correspondence Address: King & Spalding LLP, P.O. Box 889, Belmont, CA, 94002-0889, US

(73) Assignee: The Trustees of the Leland Standford Junior University, Stanford, CA (US)

(21) Appl. No.: 12/482,703

(22) Filed: Jun. 11, 2009

Related U.S. Application Data
(60) Provisional application No. 61/061,048, filed on Jun. 12, 2008.

(51) Int. Cl. A61K 39/395; A61K 38/00

(52) U.S. Cl. 424/130.1; 514/2

(57) ABSTRACT

The present disclosure is directed to methods and compositions for treating osteoarthritis and preventing osteoarthritis, by administering a compound that modulates one or more components in the complement system. In one embodiment, a compound that inhibits a component in the complement system is administered to prevent, delay the progression of, or treat osteoarthritis. In other embodiments, compounds with specific inhibition of a component in a particular pathway in the complement system, such as the alternative, mannose binding lectin, and/or classical pathway, are administered to a subject having osteoarthritis or at risk of developing osteoarthritis.

### CROSS-REFERENCE TO RELATED APPLICATIONS

This application claims the priority to U.S. Provisional Application No. 61/061,048, filed Jun. 12, 2008, incorporated herein by reference in its entirety.

### STATEMENT REGARDING GOVERNMENT INTEREST

This work was supported by the National Institutes of Health (NIH) National Heart Lung and Blood Institute (NHLBI) Proteomics Contract N01-HV-28183. The federal government has certain rights in the invention.

### TECHNICAL FIELD

The present disclosure is directed to methods and compositions for treating and/or preventing osteoarthritis.

### BACKGROUND

Osteoarthritis (OA) is the most common joint disorder in the world. The morbidity and health costs attributed to OA are substantial, and are anticipated to increase as the population ages. Although the joint is a complex structure comprised of multiple tissues that are perturbed in OA, the central lesion in all cases appears to be breakdown of the articular cartilage. This deterioration leads both directly and indirectly to pain and dysfunction, and atrophy of surrounding muscles often follows, which results in a decrease in mobility.

The medical arts recognize two types of OA, idiopathic (primary) OA and secondary OA (UpToDate, 16.1 (2008), www.uptodate.com). Idopathic OA is the term used when there is no identifiable cause; it is often associated with aging, and it can be localized to the hands, feet, knee, hip or spine. A generalized form of idiopathic OA affects 3 or more joint areas, including those involved in localized OA as well as the shoulder, sacroiliac, ankle, wrist and temporomandibular joints.

Secondary OA arises when specific conditions or events initiate the development of OA, including both isolated and repetitive trauma, ligament injury or deterioration, joint instability, congenital disorders, developmental disorders, genetic alterations, crystalline arthropathies (calcium pyrophosphate dehydrate deposition disease (CPPD)) and gout, hemearthrosis, autoimmune arthritis (rheumatoid arthritis, psoriatic arthritis, etc.), septic arthritis, osteonecrosis, Paget's disease, and other diseases including diabetes mellitus, hypothyroidism, acromegaly, and neuropathic arthropathy. Frostbite injuries have also been attributed to increased risk of developing OA. OA arising following trauma-induced cartilage and ligamentous injuries can be referred to in the art as posttraumatic OA.

A more aggressive form of OA, termed “erosive OA”, affects a small percentage of patients. Erosive OA is characterized clinically by extensive erosive and osteophytic changes at the distal interphalangeal and proximal interphalageal joints, and can also involve other joints.

Both primary and secondary OA are characterized by loss of proteoglycan content from the cartilage. Without the protective effects of the proteoglycans, collagen, the other major molecular component of cartilage, is more susceptible to degradation and thus all of the structural elements of the tissue are affected. Low-grade inflammation can also occur, particularly as breakdown products from cartilage are released into the synovial space, and the cells lining the joint attempt to remove them. Outgrowths of newly formed bone, called “spurs” or osteophytes, can form on the margins of the joints, potentially as a response to attempt to provide stability to the failing articulation. These bone and cartilaginous changes, together with any inflammation present, can be both painful and debilitating.

Stiffness and chronic pain associated with OA affect overall body mobility, as the specific joints that are most commonly affected including the knees, hips, spine, and hands. Typically these symptoms are made worse by use of the affected joint, and patients are often quite uncomfortable by the end of the day and during the nighttime.

In contrast, rheumatoid arthritis is an inflammatory synovitis that is characterized by growth of the synovial lining to form an inflammatory tissue mass known as “pannus,” and symptoms are often improved as the affected joint is “warmed up” by use.

Risk factors for OA. Multiple risk factors for OA have been identified, including advanced age, female sex, obesity, lack of osteoporosis, occupation, sports activities, previous injury, muscle weakness, hemearthrosis, proprioceptive deficits, genetic elements, amputation, acromegaly and calcium-pyrophosphate crystal disease(CPPD) (UpToDate, 16.1 (2008), www.uptodate.com). Increasing age is a major risk factor for OA, with only 0.1% of individual 25-34 years old exhibiting OA while >80% of individuals older than age 55 exhibit OA (Brandt, K. et al., Textbook of Rheumatology, 5th edition, Kelley, W N, Harris Jr, E D, Ruddy, S, Sledge, C E (Eds), W. B. Saunders, Philadelphia, p. 1383, (1997)). Obesity is a significant and modifiable risk factor for OA involving the knee, and to a lesser degree the hands and other joints (Hartz, A. J. et al., J Chronic Disease 39(4):311-9, (1986)). Occupations associated with increased use and/or microtrauma to the joints have been associated with development of OA, including dock workers, carpenters, field workers and other occupations in which there are repetitive mechanical stresses exerted on the knee, hip, hands or other joints. Certain sports are also associated with increased rates of OA in specific joints, and examples include boxing (carpometacarpal joints), gymnastics (shoulder, wrist and elbow joints), ballet dancing (talar joints), football (knee and ankle joints), and parachuting (ankles, knees and spine). Unilateral amputation of one lower extremity results in increased weight exerted on and thereby increased development of OA in the knee of the intact lower extremity. Altered propioception may contribute to the development of OA through multiple mechanisms, including reduction in protective periarticular muscle control important for normal joint alignment, alterations in quadriceps strength, as well as alterations in inflammatory responses.

Prior injury and/or joint abnormality represents a significant risk factor for the development of OA (UpToDate, 16.1 (2008), www.uptodate.com). Hip dislocation or congenital dysplasia increases the risk of later developing hip OA. An association exists between knee ligament and meniscus injuries, as well as surgical meniscus removal, with the subsequent development of OA. In addition to the meniscus and/or ligament injury reflecting prior injury to the joint, such abnormalities destabilize and alter the physiologic function of the joint in such a way that likely results in increased microtrauma and subclinical damage to the joint. In older patients, joint injury or knee surgery is associated with an even a greater risk for the development of OA in the affected joint. Meniscal tears and other abnormalities are common in patients with OA of the knee, yet only a minority of such patients recall prior knee trauma. Patients with OA involving the hand joints are at increased risk for developing OA in an operated knee. It is possible that surgical instrumentation of a joint, including arthroscopic manipulation of the knee, could result in microtrauma and thereby contribute to subsequent risk of developing OA in the operated joint. Other events that might constitute and/or contribute to joint injury include hemearthrosis (bleeding into the joint), septic joints joint infections), drug and toxin reactions that affect joint(s), and inflammatory diseases that affect the joint(s).

Diagnosis of OA. A diagnosis of osteoarthritis is made based on the presence of typical symptoms, physical examination, laboratory tests, and imaging studies. None of the clinical features of OA is entirely sensitive and specific, and is thus not necessarily predictive for OA. Overall, the more clinical features present, the more likely the diagnosis of OA. Cartilage loss, subchondral (“below cartilage”) sclerosis, subchondral cysts, narrowing of the joint space between the articulating bones, and bone spur formation (osteophytes) are the characteristic findings on x-rays and other imaging studies. With or without other techniques, such as MRI (magnetic resonance imaging), arthrocentesis and arthroscopy, diagnosis can generally be made based on the clinical history of the duration, location, the character of the joint symptoms. In addition, on physical examination the joints exhibit only minimal warmth, bony enlargement, decreased range of motion, small effusions and crepitus. A common course of OA is a slowly progressive worsening of symptoms over time, although some patients experience a stabilization of the condition. To make the diagnosis of idiopathic OA, other possible disorders need to be considered and “ruled out” (UpToDate, 16.1 (2008), www.uptodate.com). Acute severe joint pain is atypical for OA. In evaluation of a patients, synovial fluid analysis typically suggests minimal inflammation (white blood cells (WBC) <2000/mm3) and is negative for crystal examination and microbial analyses. Classical clinical criteria for the diagnosis of knee OA include knee pain plus: age >50 years old, morning stiffness <30 minutes, crepitus on active motion, bony tenderness, bony enlargement, and no palpable warmth—these criteria provide a sensitivity of 95% and a specificity of 69% (Altman R et al, Arthritis Rheum 29(8):1039-49 (1986)). OA of the hands is diagnosed based on criteria including hand pain plus >3 of the following: hard tissue (bony) enlargement of 2 or more of 10 specific joints (distal interphalangeal (DIP), proximal interphalangeal (PIP), 1^{st} carpal metacarpal (CMC)), bony enlargement of >=2 DIP joints, fewer than three swollen metacarpophalangeal (MCP) joints, and bony deformity of at least 1 of the 10 specific joints. For the diagnosis of hip OA, criteria include hip pain plus >=2 of the following features: erythrocyte sedimentation rate <20 mm/h, osteophytes on radiograph, and joint space narrowing on radiographs.

The Pathogenesis of Osteoarthritis. OA is thought to arise from genetic, metabolic, biochemical and biomechanical factors, along with a secondary component of mild to moderate inflammation, that together result in cartilage failure (UpToDate, 16.1 (2008), www.uptodate.com). Development of OA is associated with dysfunction of the metabolic, synthetic, degradative and proliferative properties of chondrocytes. Histologically, alterations in cartilage, bone, and synovium are all seen. It is proposed that physical damage to the articular cartilage, due to trauma or repetitive microtrauma, may initiate the degenerative process. In some patients, ligamentous or meniscal damage may destabilize the joint or cause misalignment, which results in biomechanical forces that induce repeated trauma or subclinical insults to the joint. In response to injury and/or insult, evidence suggests that chondrocytes enter a dysregulated repair process characterized in part by release of degraditive enzymes that subsequently break down cartilage. In a healthy joint, chondrocytes are responsible for a continuous process of remodeling and repair of cartilage (in an analogous fashion to the role of osteoblasts and osteoclasts in maintaining bone). In OA, these degradative and synthetic functions of chondrocytes become imbalanced such that degradative processes dominate, resulting in cartilage breakdown. Further, multiple additional factors become involved, including dysregulation of mechanotransduction, cytokines, proteases, and other factors that all contribute to progressive cartilage failure.

Multiple proteases are thought to play a central role in the breakdown of cartilage in OA. These proteases include metalloproteases, zinc-dependent enzymes of 3 general classes: collagenases, stromelysin, and gelatinase (UpToDate, 16.1 (2008), www.uptodate.com). Collagenases include collagenase-1 (matrix metalloprotease I (MMP-1), collagenase-2 (MMP-8) and collagenase-3 (MMP-13), which are expressed at low levels in healthy joints and are significantly upregulated in OA, where they likely contribute to cartilage breakdown. Inflammatory cytokines and other molecules contribute to the activation of collagenases. Stromelysin (MMP-3) is produced by a variety of synovial cells, and activates collagenases and degrades collagen type IX and other collagen fragments. Gelatinase A (MMP-2) cleaves type I collagen. These metalloproteases, along with other proteases, are detected near the surface of cartilage in OA. Tissue inhibitors of metalloproteases (TIMPs) are also detected in synovial joints, but are insufficiently produced and activated in OA to negate metalloprotease-mediated cartilage degradation.

A low level of inflammation is observed in OA synovial fluid, in which there is typically 200-2000/mm3 white blood cells (WBCs) along with low-level elevations of inflammatory cytokines. Two of the inflammatory cytokines elevated in OA synovial fluid include interleukin-1 (IL-1) and tumor necrosis factor alpha (TNF). IL-1 is a catabolic cytokine that contributes to cartilage breakdown (UpToDate, 16.1 (2008), www.uptodate.com) by inhibiting the synthesis of articular cartilage, promoting the synthesis of fibrocartilage, and activating stromelysin and collagenases. In animal models, intraarticular delivery of IL-1 results in cartilage damage similar to that observed in OA. TNF plays a dominant role in activating the degradative processes that lead to joint and cartilage destruction in RA, and may also contribute to the pathogenesis of OA. Other inflammatory cytokines likely also mediate the activation of catabolic pathways that contribute to cartilage breakdown in OA. In addition to the catabolic cytokines IL-1, TNF and others, certain anabolic cytokines including insulin-like growth factor 1 (IGF-1) and transforming growth factor beta (TGF-beta) are upregulated in OA.

The Complement System. The complement system is one of the primary systems by which the body recognizes pathogens and foreign antigens (Holers V. M. et al., Mol Immunol., 41(2-3):147-52, (2004); Fearon, D. T. et al., Science. 272(5258):50-3 (1996)). FIG. 1 provides a simplified schematic overview of the complement system and pathways, and as seen, complement may be activated by any of three independent initiation pathways: the classical pathway, the lectin pathway, and the alternative pathway. The classical pathway of complement activation depends on conserved sequences in the Fc regions of immunoglobulins for activation (adaptive immune response), while the other two pathways, termed the alternative, and mannose binding lectin (MBL) or lectin pathways, can be activated by spontaneous C3 hydrolysis and binding of microbial carbohydrate motifs, respectively, in the absence of antibodies. The pathways subsequently converge on C3 with the formation of a central “C3 convertase”. Activation of this regulatory complex then mediates inflammation through generation of anaphylatoxins C3a and C5a and the membrane attack complex (MAC). The MAC is composed of a complex of five complement proteins (C5b, C6, C7 and C8) which are assembled in sequence and bind to the outer surface of a plasma membrane of cells, and many copies of C9 that form a ring which traverses through the membrane (Tschopp, J. et al., Nature, 322(6082):831-4 (1986); Rosado, C. J. et al., Science, 317(5844):1548-51 (2007)).

While the alternative complement pathway activates complement to opsonize and kill pathogens, it can also mediate tissue injury. The alternative pathway does not require binding of a specific antibody for activation, and its activation is contributed to by the spontaneous hydrolysis of C3, termed “tickover” which results in a form of C3 designated C2(H2O) (Thurman, J. M. et al., Arthritis Rheum., 48:3304-7 (2006)). C3(H2O) is bound by factor B, which is itself cleaved by factor D and results in the generation of the “Alternative Pathway Initiation C3 Convertase”, which cleaves C3 into C3b and C3a. The alternative pathway is also engaged as an “amplification loop” when C3b that is formed by any of the three pathways then binds to factor B. Factor B is cleaved by factor D to form the “Amplification Loop C3 Convertase” C3bBb, which cleave further C3 molecules into C3b and C3a. The alternative pathway C3 convertases form unstable complexes that can be stabilized by the binding of properdin. Following generation of C3 convertase, the complement system utilizes the same pathway independent of the activating mechanism (classical, MBL, alternative). Recently, the alternative pathway has been demonstrated to mediate pathologic complement activation in the absence of both the classical and MBL pathways (Banda, N. K. et al., J. Immunol. 177. 1904-1912 (2006); Banda, N. K. et al., J. Immunol., 179(6):4101-9 (2007); Hietala, M. A. et al., Eur. J. Immunol., 34:1208-1216 (2006)).

Because of tickover and the detrimental effects of activated complement, multiple regulatory proteins and mechanisms have evolved (Thurman, J. M. et al., Arthritis Rheum., 48:3304-7 (2006); Song, W. C., Autoimmunity, 39(5):403-10 (2006)). These natural complement inhibitors and regulatory proteins prevent the activation of the complement system on host cells, and include: (i) complement receptor 1 (CR1 or CD35) and DAF (decay accelerating factor or CD55), which compete with factor B for binding with C3b and block the alternative pathway, as well as similarly block the classical pathway C4b from interacting with C2, (ii) factor I, a plasma protease that cleaves C3b and C4b into their inactive forms to block formation of the convertases, and (iii) factor H which can compete with factor B, displace Bb from the convertase, act as a cofactor for factor I, and bind C3b that is already bound to cells. CD59 is a complement regulatory protein that inhibits MAC (C5b-9).

Treatment of Osteoarthritis. The treatment of OA is currently focused on controlling pain and swelling, improving quality of life, and minimizing disability (UpToDate, 16.1 (2008), www.uptodate.com). Non-pharmacologic therapy includes weight loss, physical therapy and orthotics/prosthetics. There are no disease-modifying drugs available at this time, so pharmacologic therapy is primarily focused on control of pain with acetaminophen, non-steroidal antiinflammatory drugs (ibuprofen, naproxen, indomethacin, etc), cyclooxygenase-2 selective inhibitors (celecoxib, etoricoxib, etc), and other non-opiate and opiate analgesics. Intraarticular corticosteroid injections provide benefit in some patients.

It has been determined for the first time that inhibition of complement pathways can be used to treat osteoarthritis. Moreover, a subject at risk of developing or currently suffering from osteoarthritis may benefit clinically from administration of or treatment with a therapeutically effective amount of a compound that inhibits the alternative and/or classical complement pathway. Such treatment may also be effective in preventing, delaying or reducing the probability of development of osteoarthritis in a subject at risk or developing osteoarthritis or currently having osteoarthritis.

The foregoing examples of the related art and limitations related therewith are intended to be illustrative and not exclusive. Other limitations of the related art will become apparent to those of skill in the art upon a reading of the specification and a study of the drawings.

### BRIEF SUMMARY

The following aspects and embodiments thereof described and illustrated below are meant to be exemplary and illustrative, not limiting in scope.

In one aspect, a method for treating osteoarthritis is provided, by administering to a subject having osteoarthritis, a complement inhibitor compound, or a functional equivalent thereof, in a therapeutically effective amount is provided.

In other aspects, methods for treating a subject at risk of developing osteoarthritis or for slowing the progression of osteoarthritis in a subject, are provided The methods comprise administering to the subject a compound that inhibits a specific molecule, or one or more specific molecules, in a complement pathway.

In yet another aspect, a method of treating a subject suffering from or at risk of developing osteoarthritis is provided, wherein the subject is administered a compound that inhibits one or more specific molecules in a complement pathway, provided that the subject is not otherwise in need of treatment with a compound that inhibits one or more specific molecules in a complement pathway.

In one embodiment, the method comprises administering a complement inhibitor to a subject at risk of developing osteoarthritis. In another embodiment, the method comprises administering a complement inhibitor to a subject at risk of developing post-traumatic osteoarthritis. In yet another embodiment, the method comprises administering a complement inhibitor to a subject currently having osteoarthritis.

In one embodiment, the compound is a polypeptide. In another embodiment, the polypeptide is a fusion protein. In another embodiment, the compound is an antibody or functional fragment thereof In yet another embodiment, the polypeptide or antibody is purified from plasma or produced recombinantly in prokaryotic cells, eukaryotic cells or transgenic animals.

In one embodiment, the compound is a small molecule.

In another embodiment, the complement inhibitor is an inhibitor of a molecule in the alternative complement pathway. In yet a further embodiment of the method, the complement inhibitor compound is an inhibitor of a molecule in the classical complement pathway. In still another embodiment, the complement inhibitor compound is an inhibitor of a molecule in both the alternative and classical complement pathways. In yet a further embodiment, the complement inhibitor is an inhibitor of a molecule in the mannose-binding lectin (MBL) pathway. In yet a further embodiment, the complement inhibitor is an inhibitor of a molecule in the alternative, classical and/or MBL pathway.

In one embodiment the complement inhibitor compound is an inhibitor of C5, C5a, or C5b. In a preferred embodiment, the compound is a specific inhibitor of C5, C5a, or C5b. In another preferred embodiment, the complement inhibitor compound is a polypeptide or a small molecule compound that inhibits C5, C5a, or C5b. In yet another preferred embodiment, the inhibitor is an antibody that binds specifically to C5. In yet another preferred embodiment, the inhibitor is a human monoclonal antibody against complement component C5, including eculizumab, pexelizumab or another anti-C5 antibody.

In yet a further embodiment the complement inhibitor compound is an inhibitor of C3 or C3 convertase. In a preferred embodiment, the compound is a specific inhibitor of C3 or C3 convertase. In yet another preferred embodiment, the complement inhibitor compound is a polypeptide, antibody or a small molecule compound that inhibits C3 or C3 convertase.

In yet a further embodiment the complement inhibitor compound is a potentiator of factor H. In a preferred embodiment, the compound is a specific fragment of Factor H delivered to the joint. In yet another preferred embodiment, the complement inhibitor compound is a polypeptide, antibody or a small molecule compound that potentiates Factor H. In yet another preferred embodiment, the complement inhibitor consists in part of a monoclonal antibody specific for Factor H that promotes binding to the cartilage. In yet another preferred embodiment, the monoclonal antibody is an isolated human monoclonal antibody.

In another embodiment, the complement inhibitor compound is an inhibitor of the membrane attack complex.

In another embodiment the complement inhibitor compound is an inhibitor of proteases involved in the complement system. In a preferred embodiment, the complement inhibitor is C1-INH. In yet another preferred embodiment, the complement inhibitor is C1-INH purified from plasma or produced recombinantly in transgenic animals. In some embodiments, the C1-INH is recombinant human C1 inhibitor or functional equivalent thereof. In another embodiment, the complement inhibitor is a soluble complement regulator. In a preferred embodiment, the complement inhibitor is soluble CR1 (sCR1), or analogues thereof.

In other embodiments, the complement inhibitor compound is a CR2-Factor H fusion protein or a CR2-Crry fusion protein.

In other embodiments, the complement inhibitor compound is a small molecule. In yet other embodiments, the small molecule inhibits C5a or C3a.

In other embodiments, the complement inhibitor compound is a compound that prevents cleavage of C2, C3, C4, or C5.

In other embodiments, the complement inhibitor compound is a Vaccinia complement control protein (Vaccinia CCP).

In other embodiments, the complement inhibitor compound is a decay-accelerating factor (DAF), a soluble decay-accelerating factor (sDAF), a membrane cofactor protein (MCP), a soluble membrane cofactor protein (sMCP), a fusion protein comprising sMCP fused to DAF (sMCP-DAF), CD59, a soluble CD59 protein (sCD59), or a fusion protein comprising DAF and CD59 (DAF-CD59). In yet other embodiments, the compound is an MCP-DAF fusion protein. In still other embodiments, the protein is CAB-2.

In other embodiments, the complement inhibitor compound is a variant or mutant C5a protein.

In other embodiments, the complement inhibitor compound is an antibody or functional fragment thereof that specifically binds C5, C3, C5a, C3a, C4a, C6, C7, C8, C9, factor B factor D, properdin (factor P), CD20, CD38, C5 receptor (C5R) or C5a receptor (C5aR).

In yet other embodiments, the antibody that specifically binds the C5 receptor is neutrazumab.

In yet other embodiments, the antibody that specifically binds C5 is eculizumab.

In yet other embodiments, the antibody that binds CD38 is HuMax-CD38.

In yet other embodiments, the complement inhibitor compound is eculizumab.

In other embodiments, the complement inhibitor compound is a C5aR antagonist selected from the group consisting of N MeFKPdChaWdR and F-(OpdChaWR)C5aR.

In other embodiments, the complement inhibitor compound is an RNA aptamer. In yet other embodiments, the aptamer selectively binds and inhibits C5.

In other embodiments, the complement inhibitor compound is a C3 inhibitor peptide or a functional analog thereof.

In other embodiments, the complement inhibitor compound is BCX-1470, FUT-175, K-76, recombinant human mannose-binding lectin (rhMBL), APT070, TNX-234, TNX-558, TA106, complement component 4 binding protein (C4bp), Factor H, Factor I, carboxypeptidase N, vitronectin, clusterin, JSM-7717, JPE-1375,or OmCl protein.

In other embodiments, the complement inhibitor compound inhibits C5, C3, C5a, C3a, C4a, C6, C7, C8, C9, factor B factor D, properdin (factor p), CD20, CD38, C5 receptor (C5R), C5a receptor (C5aR), C1q, C1, C1r, or C1s.

In another embodiment, the method further comprises administering to the subject a further therapeutic treatment. In various embodiments, the further therapeutic treatment comprises administration of an active agent, such as an anti-inflammatory agent, an analgesic, or a steroid. In other embodiments, the further therapeutic treatment is a physical therapy, exercise or a local heat treatment. In one embodiment, when the further therapeutic treatment is an active agent, the anti-inflammatory agent is a non-steroidal anti-inflammatory agent or a cyclooxygenase-2 selective inhibitor, the analgesic is a non-opioid analgesic, or the steroid is a corticosteroid drug.

### BRIEF DESCRIPTION OF THE DRAWINGS

FIG. 1 illustrates pathways of the complement system, which include the classical pathway, the mannose-binding lectin (MBL) pathway, and the alternative pathway.

FIG. 2 shows a model of the development of osteoarthritis in mice over 12-16 weeks following surgical ligation of the stifle ligament and medial meniscectomy.

FIG. 3A is a photomicrograph image of a toluidine-blue stained stifle joint tissue section from a C57BL/6 (B6) wildtype mouse one week after ligation of the stifle ligament and medial meniscectomy.

FIG. 3B is a photomicrograph image of a toluidine-blue stained stifle joint tissue section from a B6 wildtype mouse sixteen weeks after ligation of the stifle ligament and medial meniscectomy.

FIG. 4A is a photomicrograph image of a toluidine-blue stained stifle joint tissue section from a C57BL/10 (B10) wildtype mouse sixteen weeks after ligation of the stifle ligament and medial meniscectomy.

FIG. 4B is a photomicrograph image of a toluidine-blue stained stifle joint tissue section from a C5-deficient mouse in the B10 background, sixteen weeks after ligation of the stifle ligament and medial meniscectomy.

FIG. 5A is a graph showing extent of osteoarthritis development (OA Score) in left (L; operated) and right (R; unoperated) stifle joint tissue harvested from C5-deficient mice (C5 KO) and wildtype (WT) mice four months after surgery on the left (L) stifle joint only.

FIG. 5B are photomicrographs of representative toluidine-blue stained stifle joints harvested from WT and C5deficient mice at serial time points (2, 4, 8 and 12 weeks) following surgical induction of osteoarthritis (only the operated joints are shown).

FIG. 5C is a graph showing the severity of degenerative arthritis (OA Score, plotted on the Y axis) at serial time points in groups of WT and C5-deficient (C5−) mice at serial time points following surgical induction of osteoarthritis.

FIGS. 6A-6C show the use of the Noldus CatWalk system to analyze gait in rodents.

FIG. 7A shows the frequency of gait patterns used by each of wildtype mice and C5-deficient mice, as analyzed by the CatWalk system.

FIG. 7B shows the frequency of gait patterns (Ab stride %) at serial time points in the mice described in FIG. 5B-5C. Percent Ab stride pattern is displayed at serial time points for WT non-operated (C5+ non-operated), WT operated (C5+ operated), C5− non-operated and C5− operated mice.

FIGS. 8A-8D are bar graphs showing the extent of osteoarthritis development (OA Score) in left (L) and right (R) stifle joint tissue harvested from FcγRIIb-deficient mice (FIG. 8A), FcγRIII-deficient mice (FIG. 8B), C5aR-deficient mice (FIG. 8C), and mannose-binding lectin (MBL)-deficient mice (FIG. 8D), along with wildtype (WT) control mice, four months after surgery on the left stifle joint;

FIGS. 9A-9B are photomicrograph images of osteoarthritis cartilage obtained from the knee of a human subject, stained with an anti-C3c antibody to detect deposition of C3c on the articular surface (FIG. 9A) or stained with an isotype matched control antibody (FIG. 9B);

FIGS. 9C-9D are photomicrograph images of osteoarthritis cartilage obtained from the knee of a human subject, stained with an anti-C5b-9 antibody to detect deposition of membrane attack complexes (hereafter “MAC” or “C5b-9”) on and around chondrocytes (FIG. 9C) or stained with an isotype matched control antibody (FIG. 9D);

FIG. 10 is a photomicrograph image of paraffin-embedded remnant cartilage derived from a patient with OA at the time of arthroplasty, stained with anti-5aR antibody, to illustrate that C5a receptor is not expressed in human OA cartilage.

FIG. 11A is a graph showing the concentration of MAC (C5b-9), in ng/mL, in synovial fluid samples derived from patients with, osteoarthritis (OA SF), and healthy individuals (Healthy SF).

FIG. 11B is a graph showing the concentration of C3a, in ng/mL, in synovial fluid samples derived from patients with osteoarthritis (OA SF) and healthy individuals.

FIG. 12 is a graph showing the concentration of MAC (C5b-9), in ng/mL, production as a function of time, in minutes, when pulverized osteoarthritis cartilage (OA cart), pulverized osteoarthritis synovium (OA syn), phosphate-buffered saline (PBS) (negative control) and sepharose (positive control) were added to human serum, where production of MAC was quantitated by ELISA at the indicated times.

FIG. 13 is a graph showing the concentration of MAC (C5b-9), in ng/mL, production as a function of time, in minutes, when pulverized osteoarthritis cartilage (OA cart), pulverized non-osteoarthritis cartilage (KI cart), sepharose 4b (Seph 4b), PBS, or zymosan is added to serum.

FIG. 14A is a graph showing the concentration of the MAC (C5b-9), in ng/mL, production as a function of time, in minutes, when type II collagen (coil II), aggrecan, matrilin-3, fibromodulin, zymosan, and PBS were added to 10% human serum.

FIG. 14B is a graph showing the concentration of fibromodulin, in ng/mL, in synovial fluid samples derived from patients with osteoarthritis (OA SF) and healthy individuals (Healthy SF).

FIG. 14C is a graph showing the concentration of C3a, in ng/mL, in synovial fluid samples with low levels of fibromodulin (FM lo) and high levels of fibromodulin (FM hi).

FIG. 15 is a graph showing the concentration of MAC (C5b-9), in ng/mL, production as a function of time, in minutes, when pulverized osteoarthritis cartilage (OA cart), pulverized osteoarthritis synovium (OA syn), sepharose or PBS is added to factor-B depleted serum;

FIG. 16 is a graph demonstrating inhibition of factor B eliminates the OA cartilage-induced activation of complement, and this graph shows the concentration of MAC (C5b-9), in ng/mL, production as a function of time, in minutes, when pulverized osteoarthritis cartilage (OA cart), sepharose 4b (Seph 4b), phosphate buffered saline (PBS), or aggregated human IgG (AHG) is added, either alone or in combination with anti-Factor B antibody (anti-Bb [also referred to as anti-BB]), to serum; and

FIG. 17 is a model of complement-driven osteoarthritis pathogenesis, in which cartilage injury results in cartilage breakdown products that directly activate the complement system to induce and perpetuate osteoarthritis, with complement activation inducing expression of inflammatory cytokines and activation of degradative enzymes.

### DETAILED DESCRIPTION

### I. Definitions

As used in this specification, the singular forms “a”, “an” and “the” include plural references unless the context clearly dictates otherwise.

Compounds useful in the methods are described herein and include variations of their pharmaceutically acceptable forms, including isomers such as diastereomers and enantiomers, salts, solvates, and polymorphs, as well as racemic mixtures and pure isomers of the compounds described herein, where applicable.

“Optional” or “optionally” means that the subsequently described circumstance may or may not occur, so that the description includes instances where the circumstance occurs and instances where it does not.

The terms “subject”, “individual”, or “patient” are used interchangeably herein and refer to a vertebrate, preferably a mammal. Mammals include, but are not limited to, humans.

A “cartilage component”, as used herein, refers to proteins, proteoglycans, glycoproteins, glycolipids and other molecules found in cartilage. Exemplary cartilage components include fibromodulin, type II and other collagens, matrilin 3, and aggrecan.

A “complement inhibitor,” as used herein, refers to a molecule that inhibits the activity of a complement molecule that acts in, or has an indirect interaction with, the classical complement pathway, the alternative complement pathway, or the MBL (mannose binding lectin) complement pathway. Alternatively, a complement inhibitor refers to a molecule that, when administered to an organism or cell, results in a phenotype that indicates inhibition of the activity of a complement molecule, and in one embodiment is a molecule that results in a phenotype that indicates specific inhibition of the activity of a specific target complement component molecule. Exemplary complement inhibitors are known in the art (see, for example, Ricklin, D. et al., Nat. Biotech., 25(11):1265-1275 (2007); Haynes D. et al, Biochem. Pharmacol., 60:729-33 (2000); Huber-Lang M. et al., FASEB J., 16:1567-74 (2002); PCT Publication No. WO 02/49993A2; U.S. Patent Publication No. 2007/0141573; U.S. Patent Publication No. 2007/0274989; U.S. Patent Publication No. 2005/0265995) and are set forth infra.

The term “inhibition” or “inhibit” refers to a reduction of activity.

The terms “therapeutically useful amount” and “therapeutically effective amount” refer to an amount of a compound or agent that effectively modulates a complement pathway to achieve the desired effect in terms of treating a disease, condition, or achieving a biological occurrence.

The term “antibody” herein is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

“Antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies (Zapata et al., Protein Eng. 8(10): 1057-1062 (1995)); single-chain antibody molecules; and multispecific antibodies formed from antibody fragments.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

“Fusion” protein refers to a protein constructed by appending at least one contiguous portion of a first protein to at least one contiguous portion of a second, different protein. In an analog of a fusion protein, either or both of the contiguous portions comprising the chimera, one or more amino acid residues may be substituted, deleted or added to the native sequence of such constituent. A fusion protein may or may not comprise a “linker” sequence.

A “small molecule” herein is defined as having a molecular weight below about 500 Daltons.

All patents and patent applications cited herein, whether supra or infra, are hereby incorporated by reference in their entirety.

### II. Methods of Treatment

The methods described herein are directed to treating persons with osteoarthritis or at risk of developing osteoarthritis. The methods are also directed to slowing or preventing the progression of osteoarthritis in persons suffering from osteoarthritis or at risk of developing osteoarthritis. These methods are achieved by administering to the subject a compound that modulates a component in the complement system. In a preferred embodiment, a compound that inhibits a complement component is administered to the subject. As will now be described, studies were conducted to show that modulation of a component in the complement system is effective to treat osteoarthritis and/or prevent or delay development and/or progression of osteoarthritis.

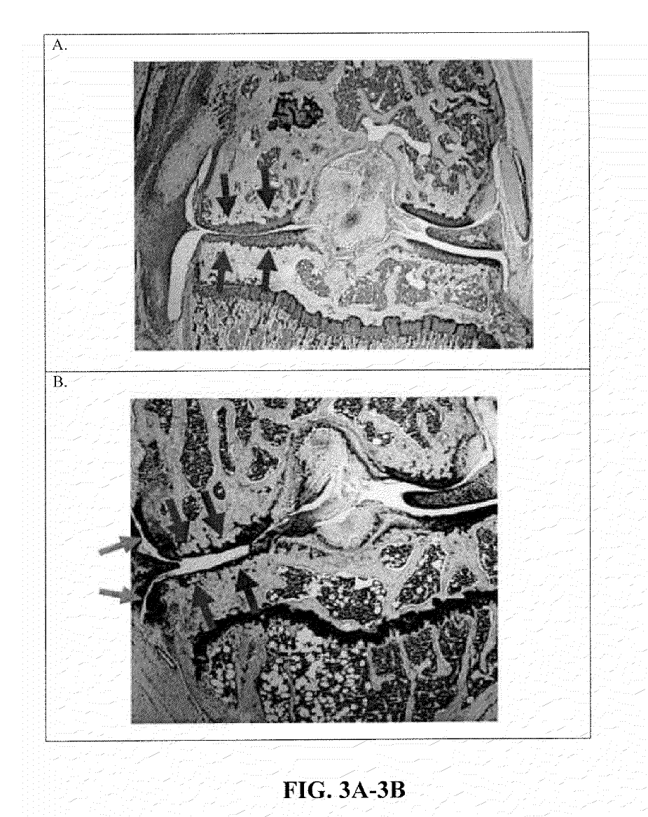

In a first study, detailed in Example 1, a murine model for surgical induction of osteoarthritis was used to evaluate the role of complement components in osteoarthritis, by performing the surgery on wildtype and knock-out mice. As illustrated in FIG. 2, surgical ligation of the stifle ligament and medial meniscectomy of the stifle joint (rodent knee equivalent) results in the development of osteoarthritis. Surgical ligation was performed on C57BL/6 (B6) wildtype mice and on complement component 5 (C5) deficient mice (“C5-knock out” mice, generated as described in Example 1), and at one week and sixteen weeks post-ligation, the stifle joints were harvested and prepared for visual inspection. FIG. 3A shows the stifle joint one week following stifle ligament ligation and medial menisectomy in a B6 wildtype mouse. The medial meniscus is absent, as expected, but the articular cartilage is intact and proteoglycan content normal (arrows). FIG. 3B shows the stifle joint sixteen weeks following surgery in a B6 wildtype mouse. Extensive degeneration of the articular cartilage in the medial compartment (arrows) is observed, and development of osteophytes (arrows at far left of image) is evident.

The surgically-induced osteoarthritis shares multiple features with human osteoarthritis on histologic analysis, including proteoglycan and cartilage loss (FIG. 3B) and ectopic bone formation consistent with osteophytes (FIG. 3B). Mice sacrificed one week following surgical stifle ligament ligation and medial meniscectomy exhibited no evidence of proteoglycan loss or cartilage degeneration (FIG. 3A), suggesting that the degenerative arthritis phenotype develops over a several month period.

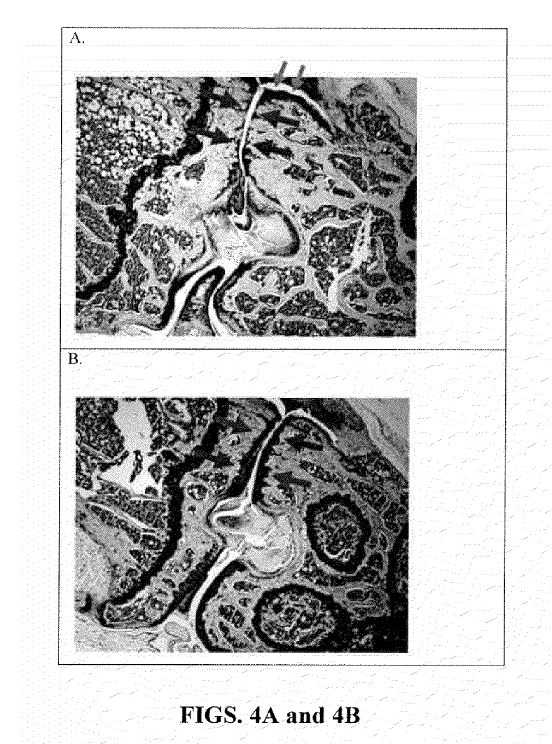

C5-deficient mice were generated, as detailed in Example 1, and subjected to surgical stifle ligament ligation to induce osteoarthritis. Sixteen weeks after surgery, the stifle joints were harvested and inspected. FIG. 4A shows a tissue section of a wildtype mouse stifle joint at sixteen weeks post-surgery, and FIG. 4B shows a tissue section of a C5-knockout mouse stifle joint at sixteen weeks post-surgery. Comparison of the images shows that in the C5-deficient mice the cartilage and proteoglycan were preserved and there was no significant development of osteophytes, establishing that osteoarthritis did not develop in C5-deficient mice. The image in FIG. 4A of the joint in wildtype mice shows extensive proteoglycan and cartilage loss (FIG. 4A, four larger arrows) and osteophyte formation (two smaller arrows at edge of image).

It will be appreciated by one of skill in the art that mice deficient in other components of the complement system can be obtained or generated, and tested as set forth in Example 1 to assess the role of the component in osteoarthritis and the development of osteoarthritis. Mice genetically deficient in other complement components are described in Example 2.

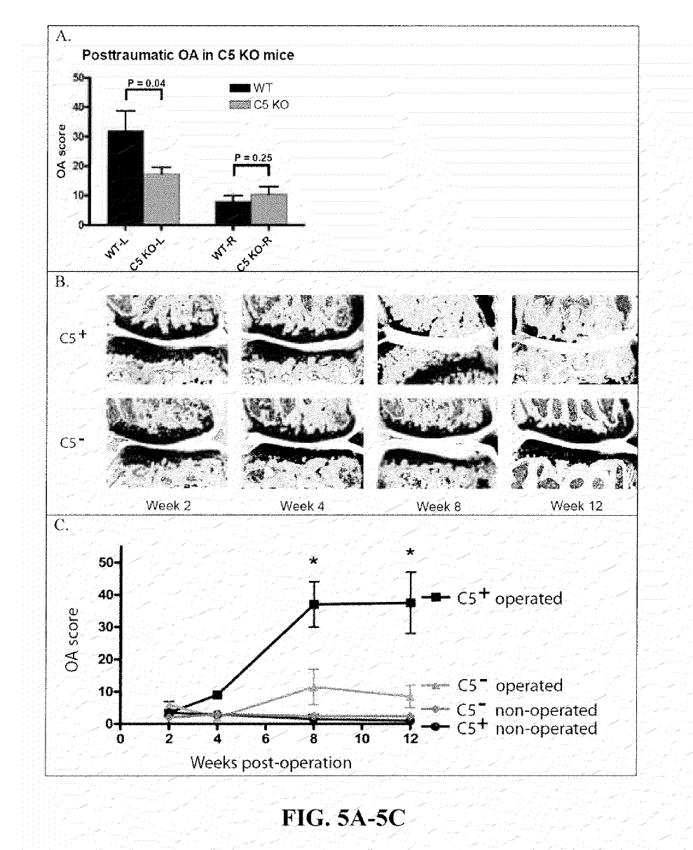

Further studies were conducted on C5-deficient mice. In this study, a quantitative scoring of the osteoarthritis phenotype in wildtype and C5-deficient mice was done. Sixteen weeks after stifle-joint ligation surgery, the stifle-joints of wildtype and C5-deficient mice were harvested and scored for the degree of posttraumatic osteoarthritis, as described in Example 3. FIG. 5A shows the results of a calculation of the OA score in C5-deficient and wildtype mice. C5-deficient mice developed statistically less severe degenerative arthritis in their surgically injured left (L) stifle joint as compared to wildtype (WT) control animals. The unoperated right (R) stifle joints exhibited no significant arthritis and no statistical differences in scores between C5-deficient and wildtype mice. As shown in FIGS. 5B and 5C, a time course experiment was performed to demonstrate that degenerative arthritis develops over time following surgical induction. This study further establishes that inhibition of the C5 component provides statistically significant protection against development of degenerative osteoarthritis.

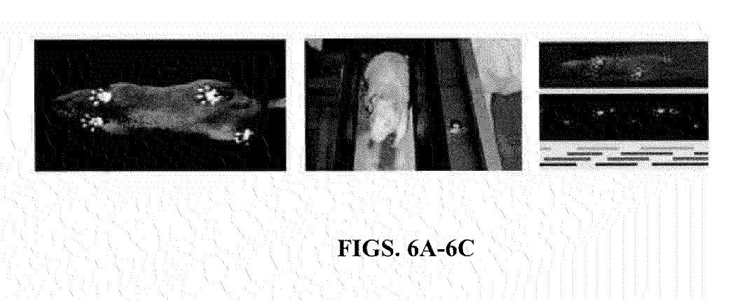

Gait analysis is another technique to monitor development and progression of osteoarthritis, as the development of osteoarthritis is correlated with loss of maintenance of normal gait. Behavioral studies were done to measure gait in wildtype and C5-deficient mice surgically induced to develop osteoarthritis. Gait analysis was performed using the Noldus CatWalk Gait Analysis System, shown and demonstrated in FIGS. 6A-6C, and which was developed to characterize neurologic deficits in mice (Gabriel A. F. et al., J. Neurosci. Methods, 163(1):9-16 (2007)). In this system mice walk on a glass catwalk, a camera captures images and quantitates pressure exhibited by each paw, and a computer analyzes the “stride pattern activity”.

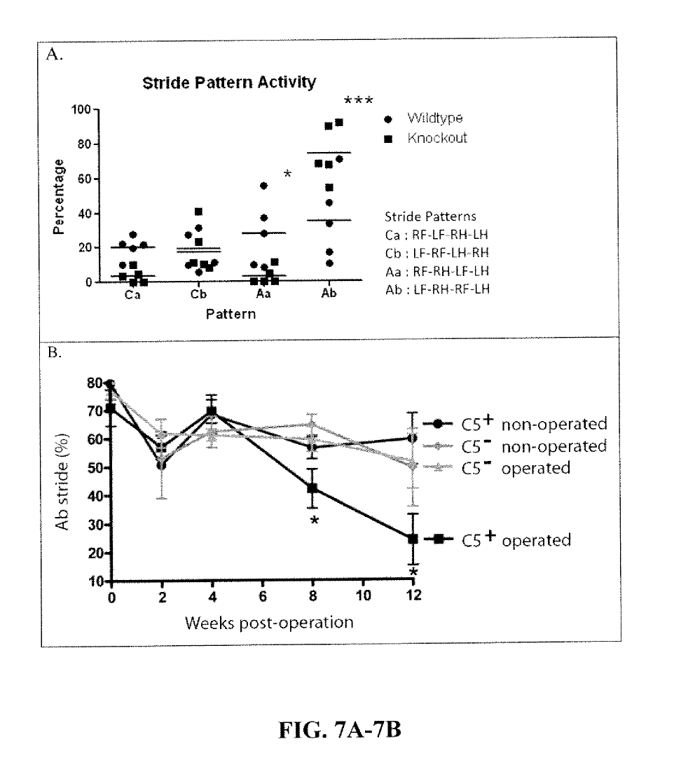

Gait analysis was obtained on C5-deficient and wildtype mice to gait analysis 16 weeks following surgical induction of osteoarthritis. As described in Example 4, each mouse walked the cat walk three times, and the CatWalk system analyzed the stride patterns. As seen in FIG. 7A, C5-deficient mice (squares) that were resistant to development of posttraumatic osteoarthritis maintained the normal “Ab” stride pattern, while wildtype mice (circles) that developed severe degenerative arthritis exhibited statistically less frequent use of the “Ab” stride pattern. Healthy mice use the stride pattern “Ab” in 70-80% of their gait, and stride patterns “Ca”, “Cb”, and “Ab” in <20% of gait. As seen in FIG. 7B, gait analysis was performed over time, to assess the degree of gait abnormality that reflects the severity of degenerative arthritis. C5-deficient mice were protected against degenerative arthritis (FIGS. 5B and 5C), and protection against osteoarthritis was associated with maintenance of the normal Ab stride pattern (FIG. 7B).

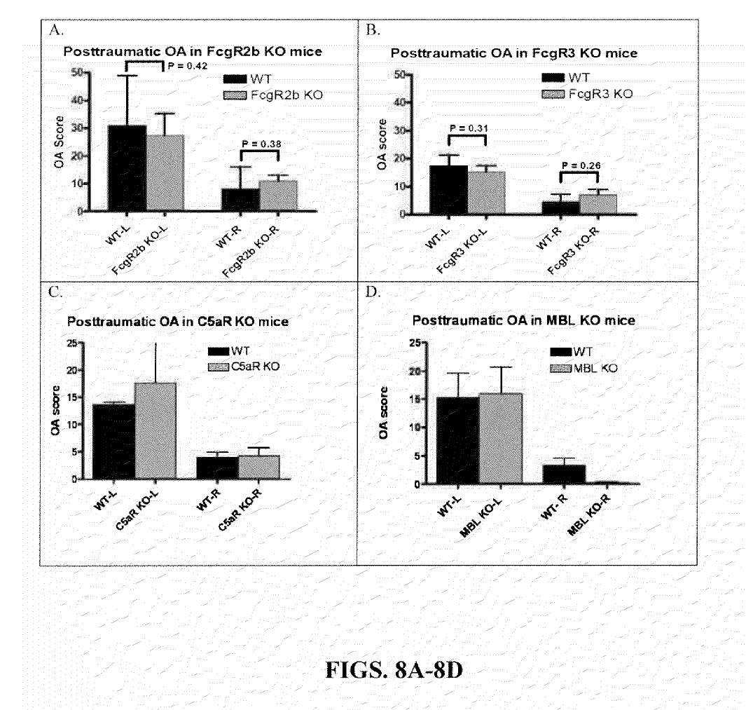

Example 5 provides an example of negative controls, where mice genetically deficient for FcγRIIb or FcγRIII, components in the classical complement pathway, are not protected from developing osteoarthritis. As shown in FIGS. 8A-8B, Ig deficient mice were also not resistant to the development of posttraumatic OA. The data of FIGS. 8A-8B demonstrate that these FcRs and their associated pathways are not central to the pathogenesis of posttraumatic OA in mice. Further, the data of FIGS. 8A-8B show that posttraumatic OA is mechanistically distinct from passive antibody transfer arthritis (induced with anti-glucose-6-phosphate isomerase (GPI) antibodies or anti-type II collagen antibodies) in which antibody and FcRs play a central role (Ji, H. et al., Immunity. 16(2):157-68 (2002); Diaz de Ståhl, T. et al., Eur J Immunol., 32:2915-22 (2002); Matsumoto, I. et al., Science, 286(5445):1732-5 (1999)).

Example 5 also demonstrates that the mannose-binding lectin (MBL) pathway is not involved in the development of osteoarthritis in this mouse model. As shown in FIG. 8, mice deficient for mannose binding lectin pathway molecules MBL-1 and MBL-2 were not protected against the development of posttraumatic OA. Taken together, these data suggest that the alternative complement pathway plays a central role in mediating post-traumatic low-grade inflammation that results in development of OA. As described above, component C5-deficient mice are protected against the development of post-traumatic OA. C5 is at a central point in the complement cascade and can be activated via the classical, mannose-binding lectin (MBP), and alternative complement pathways. It is anticipated that mice deficient for additional complement components that mediate the alternative pathway (Factor B) will be protected against development of post-traumatic OA.

It is important to delineate the downstream effector mechanisms by which joint injury-mediated activation of the complement system damages cartilage and chondrocytes. The results suggest that the MAC and not the anaphylatoxin effector pathway mediates post-traumatic cartilage injury.

Surgical joint injury may be carried out in any of several mouse strains. Development of significant posttraumatic OA has been observed in the B6, B10, and 129 SvEv backgrounds. C57BL/10 (B10) and 129 SvEv mice are readily susceptible to osteoarthritis development, which is in agreement with published results (Glasson S., Current Drug Targets, 8(2):367-76 (2007)). When significant degenerative arthritis is not observed in a specific line of genetically deficient mice, the line may be backcrossed onto the B10 or 129 SvEv background to ensure that protection against degenerative arthritis is mediated by the deficient gene product and not a resistant genetic background.

Statistical power calculations for the group sizes were calculated using the PS Power and Sample Size Calculations software program (W.D. Dupont and W.D. Plummer, Department of Biostatistics, Vanderbilt University; Version 2.1.30; http://biostat.mc.vanderbilt.edu/twiki/bin/view/Main/PowerSamplesize). Calculations accounted for the variability of surgical induction and inter-mouse differences within groups of mice in the resulting histologic OA scores (OA scores determined by a blinded examiner). Based on the aggregate variability of the surgical induction of OA in mice calculated using the results from 5 independent experiments, the following parameters were input into this program: T test sample size determination, unpaired: α=0.71 (Type I error probability), □2 =11 (difference in population means), σ=12 (within group standard deviation), m=1 (ratio of control to experimental mice), and power=0.8. PS Power and Sample Size Calculation software calculated a sample size of 4 mice per experimental arm needed to detect a true difference of 11 in the OA score with a significance level of 0.05. Based on this power calculation, a sample size of 4-5 mice per experimental arm was used.

The Student's T Test, unpaired and two tailed, performed using Microsoft Excel were used to determine statistical significance of results in the murine posttraumatic osteoarthritis model. The Student's T Test was applied to determine if differences in the histologic OA scores were statistically different between experimental arms, as well as to determine if the percentage of stride patterns as measured by the CatWalk System were statistically different between experimental arms.

The studies described above illustrate the involvement of components of the complement system in osteoarthritis and in the development of osteoarthritis. In light of these studies, it can be appreciated that modulation of one or more components of the complement system provides an approach to treating osteoarthritis and/or inhibiting the progression of osteoarthritis and/or preventing the development of osteoarthritis in persons at risk. More particularly, the in vivo data illustrates the involvement of complement component 5 in osteoarthritis, and administration of a compound effective to inhibit C5 is anticipated to be therapeutically effective to treat osteoarthritis or slow its progression or prevent its development. Compounds that inhibit components of the alternative complement pathway and/or the MAC are also anticipated to provide efficacy in this rodent model of OA.

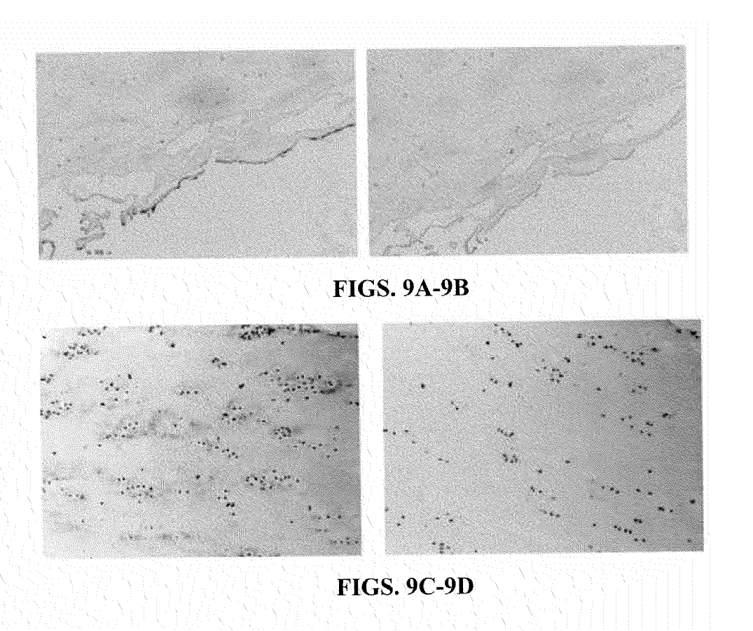

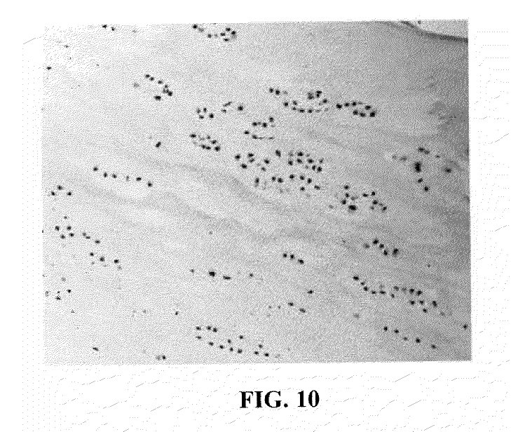

Identification of complement components involved in osteoarthritis can also be identified using in vitro techniques, as will now be described. In one study, remnant cartilage was obtained from a human patient at the time of knee arthroplasty. Immunohistochemistry was performed on the tissue, using anti-C3a antibody, anti-C5b-9 antibody (anti-MAC) antibody, and anti-5aR antibody, obtained as outlined in Example 6. The results are shown in FIGS. 9-10. The tissue stained with anti-C3 antibodies (FIG. 9A) demonstrates deposits of C3c on the surface of articular cartilage, whereas no staining was observed with the isotype matched control antibody (FIG. 9B), FIG. 9C shows that tissue stained with anti-C5b-9 (MAC) antibodies demonstrated the presence of MAC on and surrounding chondrocytes in the OA cartilage, whereas no staining was observed with the isotype matched control antibody (FIG. 9D). Immunohistochemistry did not show expression of the C5aR in OA cartilage, as seen in FIG. 10. C5a receptor induces chemoattraction and inflammation, as one of the major downstream effector pathways of the complement system. The lack of staining for C5aR in combination with the positive staining for MAC in OA cartilage (FIG. 9C) suggests that the MAC (C5b-9) is a downstream effector mediating cartilage destruction in OA.

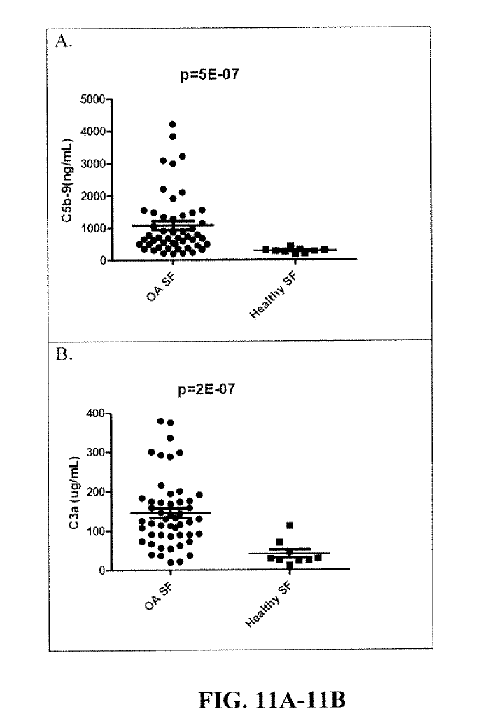

In another study, ELISA was performed to quantify levels of the MAC (C5b-9, the terminal complement complex) in synovial fluids obtained from human patients with osteoarthritis, rheumatoid arthritis, gout, and calcium pyrophosphate crystal disease, and compared to MAC levels in serum form healthy human subjects. Results are shown in FIG. 11A, where the concentration, in ng/mL, of MAC in the synovial fluid from the patients is shown. In FIG. 11B, the concentration of complement component C3a, in ng/mL, is presented. Synovial fluids from osteoarthritis (OA SF) patients exhibited a distribution of MAC and C3a levels, with approximately ⅓ of OA patients possessing high levels suggesting activation of the complement system, ⅓ intermediate levels, and ⅓ with low levels of the MAC and C3a.

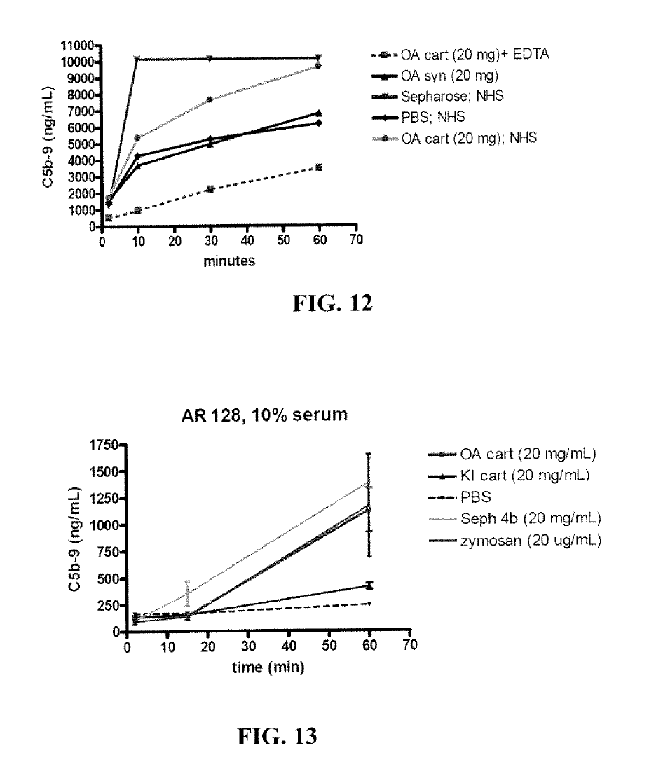

In other studies, in vitro complement activation assays were performed to identify specific cartilage components that activate the complement system, and to identify additional components of the complement system for modulation for use in the methods described herein. In these studies, various cartilage components were pulverized and added to human serum, and production of MAC was quantified by ELISA at various time points. Details are given in Example 7, and results are shown in FIGS. 12-16.

FIG. 12 shows the concentration of MAC produced when pulverized osteoarthritis cartilage is added to human serum (circles), when pulverized osteoarthritis cartilage plus EDTA is added to serum (squares), and when pulverized osteoarthritis synovial lining tissue is added to serum (triangles). As a positive control, MAC production was quantified after addition of sepharose to serum (inverted triangles), and as a negative control, MAC production was quantified after addition of phosphate buffered saline to serum (diamonds). As expected, sepharose activated the complement system to produce MAC. FIG. 12 also shows that OA cartilage activated the complement system to produce MAC (circles).

FIG. 13 shows that pulverized cartilage derived from a healthy knee joint (KI cart; closed triangles) did not result in the production of the MAC demonstrating that it did not activate the complement system. Pulverized osteoarthritis cartilage (OA cart), zymosan and sepharose 4b (seph 4b) all activated production of the MAC. Results are shown in FIG. 13. These data suggest that pulverized osteoarthritis cartilage, but not pulverized healthy cartilage, activates the complement system.

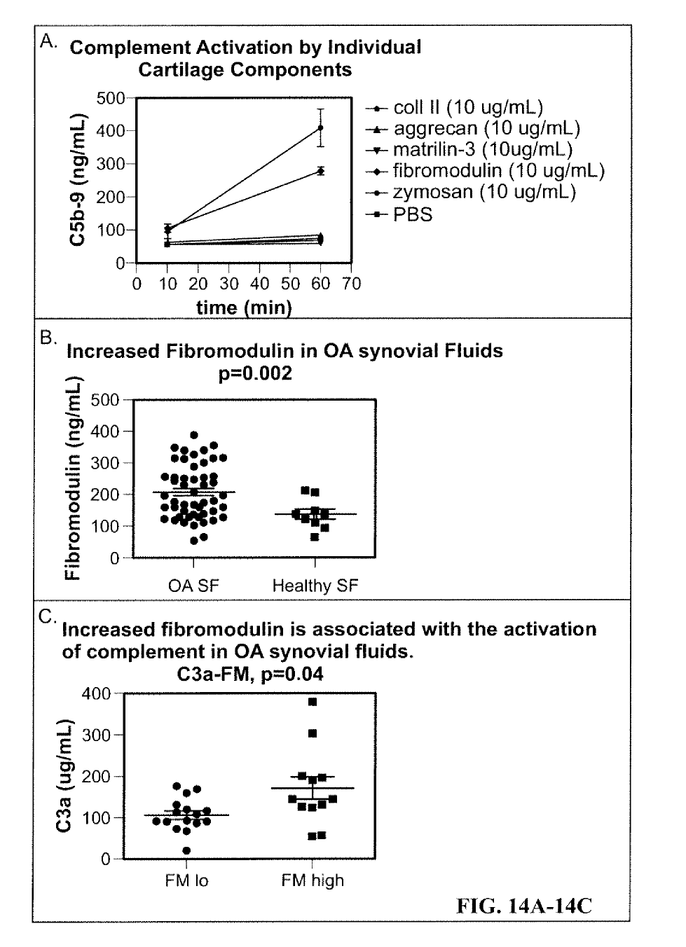

FIG. 14A shows the ability of individual cartilage components to active the complement system. Recombinant fibromodulin (diamonds) was added to 10% human serum and the production of MAC was measured as a function of time by ELISA. Similar studies were done using other cartilage proteins or proteoglycans, including type II collagen (squares), aggrecan (triangles), and matrilin-3 (inverted triangles). Zymosan (circles) and PBS (open squares) were used as positive and negative controls, respectively, for MAC production. Fibromodulin activates the complement system to produce MAC (C5b-9). FIG. 14B shows that elevated levels of the MAC (C5b-9, the terminal complement complex), measured in ng/mL, are present in synovial fluid samples derived from OA patients as compared to healthy individuals. FIG. 14C shows that in synovial fluid samples derived from OA patients, elevated levels of fibromodulin are associated with increased levels of C3a, suggesting that elevated fibromodulin is associated with the activation of the complement system.

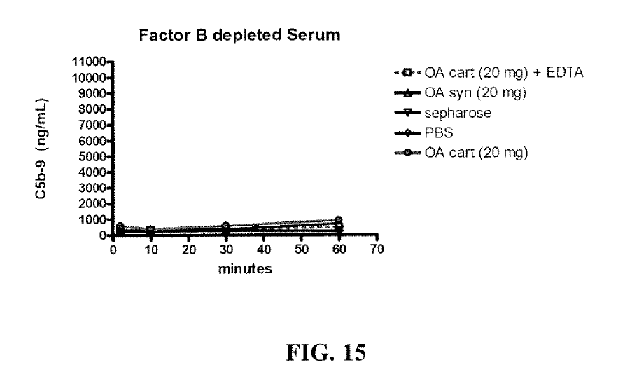

FIG. 15 shows results from a study was conducted using factor B-depleted serum, which lacks Factor B, an essential component of the alternative pathway. Cartilage components were added to the factor-B depleted serum, and production of MAC as a function of time was evaluated using ELISA. Results are shown in FIG. 15. Pulverized osteoarthritis cartilage (open squares) did not induce production of MAC (C5b-9) in factor B-depleted serum, nor did the positive control sepharose (inverted triangles) or synovial lining tissue from an osteoarthritis patient (triangles). These data suggest that pulverized osteoarthritis cartilage activates the complement system via the alternative pathway.

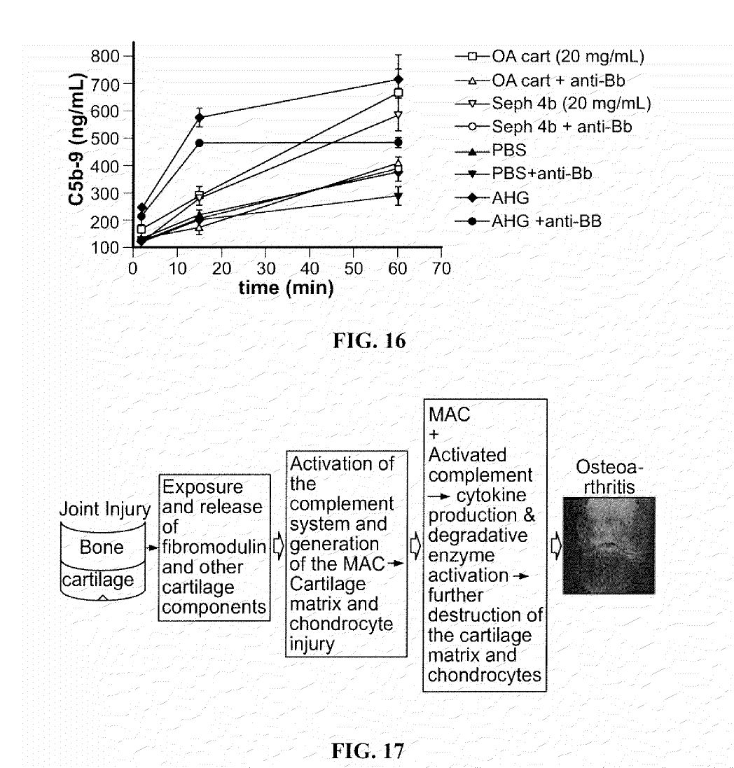

A similar study was then conducted using antibody to factor B, which inhibits its function and thus inhibits the alternative complement pathway. Normal human serum was preincubated with murine monoclonal antibody to factor B (anti-Bb), followed by addition of pulverized osteoarthritis cartilage (OA cart), PBS, Sepharose 4b (seph 4b), or aggregated human IgG (AHG). MAC was then quantified by ELISA at various time points. In the absence of anti-Bb antibody, OA cartilage, sepharose 4b, and aggregated human IgG activate complement. Activation by sepharose 4b, which occurs via the alternative pathway, is eliminated by preincubation with anti-Bb antibody. The same is observed for osteoarthritis cartilage. Anti-Bb antibody does not block activation by human IgG, which occurs via the classical pathway. Results are shown in FIG. 16.

Together, as presented in FIG. 17, these data suggest that cartilage breakdown products directly activate the alternative pathway of the complement system to induce and perpetuate osteoarthritis. Complement activation induces expression of inflammatory cytokines and activation of degradative enzymes that contribute to pathogenesis.

As mentioned above, the medical arts recognize various types of osteoarthritis, and the methods described herein are contemplated for use in treating any type of osteoarthritis. For example, the osteoarthritis can be degenerative arthritis, posttraumatic osteoarthritis, osteoarthritis arising from joint abnormalities or instability, inflammatory osteoarthritis, and erosive osteoarthritis. Treatment can be directed to patients established and symptomatic osteoarthritis, patients experiencing “pre-clinical” osteoarthritis, where progressive cartilage degeneration is evident by, for example, plain X-ray or MRI imaging of a joint, yet the patient is generally asymptomatic, as well as patients at risk of developing osteoarthritis due to injury to a joint or due to a genetic predisposition.

### III. Complement Modulator Compounds

The methods of treatment described herein comprise administering to the subject a compound that modulates one or more components of the complement system. A variety of compounds that inhibit or activate one or more components of the complement system are known in the art, and a skilled artisan will readily be able to select an appropriate compound. Examples are provided herein as merely exemplary of the compounds available.

The complement inhibitors to be utilized may include both antibodies and fusion proteins. Some exemplary antibodies, anti-C5 antibody and anti-factor B antibody, and dosage regimes are listed in Table 1.

Overall, exemplary complement inhibitors include, but are not limited to, anti-C5 antibody, anti-factor B antibody, CR2-Crry, CR2-factor H, CR1, and CR2-factor H.

Anti-mouse C5 antibody blocks the central complement component C5 and has been demonstrated to protect mice against collagen-induced arthritis (Banda, N. K. et al., Arthritis Rheum. 46: 3065-3075 (2002); Wang, Y. et al., Proc. Natl. Acad. Sci. USA 92: 8955-8959 (1995)). Anti-C5 antibodies include, but are not limited to, eculizumab (Soliris®; Alexion Pharmaceuticals, Inc.), pexelizumab (Alexion Pharmaceuticals, Inc.)

Anti-factor B antibody is a non-immunogenic, mouse anti-mouse reagent that blocks the association of factor B with C3 and has been shown to be protective in several animal models of acute and chronic inflammation. Anti-factor B antibodies are taught, for example, in U.S. Publication No. 2008/0102040 and 2008/0299114.

CR2-Crry is a murine fusion protein in which the complement inhibitor Crry (the rodent analog of human CR1) has been fused to the targeting domain of CR2 which specifically binds the long-lived C3 cleavage fragments (iC3b, C3dg and C3d) generated at sites of complement activation (Ahearn, J. M. et al., Adv. Immunol., 46:183-219 (1989); Atkinson, C. et al., J Clin Invest. 115(9):2444-53 (2005); Song, H. et al., J Immunol., 179(11):7860-7 (2007) ). Cr2-Crry fusion proteins also are taught, for example, in U.S. Publication No. 2008/0221011 and No. 2008/0267980.

CR1 attenuates complement activation by promoting dissociation of components of the C3 convertase on cell membranes.

A C1 inhibitor (C1INH), also referred to as C1 esterase inhibitor, is an inhibitor of complement C1. C1INH belongs to the superfamily of serine proteinase inhibitors and is the only inhibitor of C1r and C1s of the complement system and is the major inhibitor of factor XIIa and kallikrein of the contact system. Human C1-INH is a protein of 500 amino acids, including a 22 amino acid signal sequence (Carter et al. 1988, Euro. J. Biochem. 173; 163). Plasma C1INH is a glycoprotein of approximately 76 kDa and is heavily glycosylated, up to 26% of its molecular mass consists of carbohydrate (Perkins et al., 1990, J. Mol. Biol. 214, 751). A C1INH for use in the methods of the present invention preferably is a protein with an amino acid sequence that has at least 65, 67, 68, 69, 70, 75, 80, 85, 90, 95, 98, 99 or 100% identity with the amino acid sequence of the mature human C1INH as depicted in SEQ ID NO: 1.

CR2-Factor H is a murine recombinant fusion protein in which the complement inhibitor domain of Factor H (SCR1-5) has been fused to the targeting domain of CR2. Factor H inhibits activation of the alternative pathway, and regulates complement activation on cell surfaces though co-factor activity for Factor I-mediated C3b cleavage and decay accelerating activity against the alternative pathway C3 convertase (C3bBb). CR2-Factor H exhibits potent inhibitory activity against the alternative pathway, demonstrating 10-20-fold improvement in activity as compared to factor H itself, and possesses a prolonged in vivo half life which enables weekly dosing.

In some embodiments, there is provided a CR2-FH molecule comprising: a) a CR2 portion comprising a CR2 or a fragment thereof, and b) a FH portion comprising a FH or a fragment thereof, wherein the CR2-FH molecule is capable of binding to a CR2 ligand and wherein the CR2-FH molecule is capable of inhibiting complement activation of the alternative pathway. In some embodiments, there is provided an isolated CR2-FH molecule. In some embodiments, there is provided a composition (such as a pharmaceutical composition) comprising a CR2-FH molecule. In some embodiments, the CR2 portion and the FH portion are directly or indirectly fused to each other in the form of a fusion protein. In some embodiments, the CR2 portion and the FH portion are linked via a chemical crosslinker. In some embodiments, the CR2 portion and the FH portion are non-covalently linked. Further teachings of CR2-FH fusion constructs are provided in U.S. Publication No. 2008/0221011.

TABLE 1
Exemplary complement inhibitors and Dosing Regimens in Mice

| Inhibitor | Mechanism | Dose and route |
|---|---|---|
| Anti-C5 Ab | Blocks C5 | 750 ug/IP 2× per wk |
| Anti-factor B Ab | Blocks factor B | 1000 ug/IP 3× per wk |
| CR2-Crry | Inhibits C3 convertase | 0.25 mg IV once per wk |
| CR2-Factor H | Inhibits C3 convertase | 0.25 mg IV once per wk |
| Istoype ctrl. Ab | Control | 750 ug/IP 2× per wk |

Complement inhibitors additionally include, for example, Soluble Human Complement Receptor Type 1 (sCR1; Swift et al., Clin Diagn Lab Immunol., 1(5):585-589, (1994)), Vaccinia CCP (Vaccinia complement control protein), soluble decay-accelerating factor (sDAF), soluble membrane cofactor protein (sMCP), a fusion protein comprising sMCP fused to DAF (sMCP-DAF), soluble CD59 (sCD59), a fusion protein comprising DAF fused to CD59 (DAF-CD59) (as taught, for example, in U.S. Publication 2008/0267980), C5a mutants, Anti-C3 antibody, Anti-C5a antibody, Anti-C3a antibody, the C5aR antagonists N MeFKPdChaWdR and F-(OpdChaWR)C5aR, RNA aptamers that inhibit human complement C5 (Biesecker et al., Immunopharmacology, 42(1-3):219-230 (1999)), BCX-1470, FUT-175, K-76, thioester inhibitors, C1-INH (Cetor/Sanquin, BerinertP/CSL Behring, Lev Pharma), Rhucin/rhC1 INH (Pharming Group N.V.), sCR1/TP10 (Avant Immunotherapeutics), CAB-2/MLN-2222 (Millenium Pharmaceuticals), ofatumumab, a human monoclonal antibody that specifically binds the CD20 protein (also known as HuMax-CD20; Genmab A/S), a C3 inhibitor peptide and its functional analogs (Compstatin/POT-4; Potentia Pharmaceuticals, Inc.), a C5a receptor antagonist (PMX-53; Peptech, Ltd.), rhMBL (Enzon Pharmaceuticals), Factor D inhibitor BCX1470, sCR1-sLeX (a soluble from of CR1 that has been modified by the addition of sialyl Lewis x (sLe^{x}) carbohydrate side chains yielding sCR1sLe (TP-20; Avant Immunotherapeutics, Inc.)), APT070, which consists of the first three short consensus domains of human complement receptor 1, manufactured in recombinant bacteria and modified with a membrane-targeting amphiphilic peptide based on the naturally occurring membrane-bound myristoyl-electrostatic switchpeptide (Mirococept (Inflazyme Pharmaceuticals), TNX-234 (Tanox), TNX-558 (Tanox), an antibody or functional fragment thereof that binds factor B (TA106; Taligen Therapeutics, Inc.), an antibody that specifically binds the C5 receptor (e.g., neutrazumab; G2 Therapies, Inc.), Anti-properdin (Novelmed Therapeutics), HuMax-CD38 (Genmab A/S), a pegylated aptamer-based C5 inhibitor (ARC1905; Archemix, Inc.), and a small molecule/peptidomimetic antagonist for the C5a receptor protein (e.g., JPE-1375, JSM-7717; Jerini, Inc.), OmCl protein, compstatin and its functional analogs, C1q inhibitors, C1 Inhibitor, C1r inhibitors, C1s inhibitors, analogues of sCR1, anti-C5a receptor antibodies and small-molecule drugs, anti-C3a receptor antibodies and small-molecule drugs, anti-C4a antibodies and their functionally equivalent fragments, anti-C6, C7, C8, or C9 antibodies, anti-Factor D antibodies, anti-properdin antibodies, Membrane Cofactor Protein (MCP), Decay Accelerating Factor (DAF), and MCP-DAF fusion protein (CAB-2), C4bp, Factor H, Factor I, Carboxypeptidase N, vitronectin and clusterin, CD59, c5a receptor antagonists, and F-[oPdChaWR].

Recently, the first complement inhibitor was approved by the U.S. FDA for the treatment of paroxysmal nocturnal hemoglobinuria (PNH). This inhibitor is a monoclonal antibody against complement component C5 (eculizumab (Solaris™), Alexion Pharmaceuticals), and blocks a central component of the complement system. The most significant adverse event associated with long-term eculizumab treatment in human trials was the development of meningococcal infections, with two patients in these trials experiencing meningococcal sepsis. In addition, eculizumab treatment was associated with an increased rate of upper respiratory infections (23% of patients treated). Such infectious complications were associated with long-term systemic treatment, and it is possible that in the post-traumatic setting that: (i) a brief treatment period of several months might prevent initial activation of the complement cascade and thereby circumvent the inflammatory cycle that leads to OA, and (ii) that intraarticular administration (also for a brief period) could provide benefit. Both of these strategies could significant reduce the risk of infectious complications. It is also possible that in patients developing OA a treatment period and/or intraarticular administration could interrupt activation of the complement cascade and thereby turn-off or reduce the inflammatory cycle that results in progression of OA.

In one embodiment, a complement inhibitor, such as a recombinant anti-C5 antibody, anti-factor B antibody, CR2-Crry, or CR2-factor H, is used as a therapeutic agent to prevent development of degenerative arthritis in a person at risk. In another embodiment, a complement inhibitor is administered to a human patient who has osteoarthritis or who wishes to prevent osteoarthritis. Long-term treatment following joint injury may be provided, in some embodiments. Alternatively, a brief period of treatment following joint injury or insult may be sufficient to extinguish the development of posttraumatic OA, in other embodiments. Examples 11-16 provide additional examples of treating subjects diagnosed with OA or at risk of developing OA.

In another embodiment, the treatment methods described herein further comprise administering to the subject a second or further treatment regimen. For example, in conjunction with administration of a complement inhibitor compound, the subject can be treated with local application of heat, preferably moist heat, to ease inflammation and swelling in the joints. In other embodiments, the subject is additionally treated with diet regimen for weight control, rest, or an exercise plan, or with physical therapy of at least the affected joint(s). Regular exercise, if possible, especially in the form of walking or swimming, is contemplated, and can be combined with applying local heat before, and cold packs after exercise. Dietary supplements are contemplated as a further therapy, and can include glucosamine, chondroitin, omega-3 fatty acids, herbal supplement, antioxidants, hydrolyzed collagen, ginger, selenium, vitamins B9 and B12, bone morophogenetic proteins, and the like.

Administration of additional therapeutic agents, in combination with the complement modulating compound, is also contemplated. The additional therapeutic agent can be, in preferred embodiments, acetaminophen, non-steriodal anti-inflammatory drugs, such as diclofenac, ibuprofen and naproxen, COX-2 selective inhibitors, such as celecoxib, corticosteroids, and narcotic pain relievers, such as tramadol and opioids (hydrocodone, oxycodone and morphine).

### IV. Examples

The following examples are illustrative in nature and are in no way intended to be limiting.

### Example 1

### Animal Model of Osteoarthritis and Studies in Wildtype Mice and Complement Component 5 (C5) Knock-Out Mice

Complement component 5 (C5) deficient mice were generated by backcrossing the C5-deficient DBA/2 strain onto a C57BL/10 background to give the B10D2oSn-J strain (Mastellos, D. et al., J. Immunology, 166(4):2479-86 (2001)). The B10D2oSn-J strain bears a 2-bp (TA) deletion in an exon near the 5′ end of the C5 gene. This deletion results in the expression of a truncated protein that accounts for the C5 protein deficiency. C57BL/6 (B6) wildtype mice were obtained from a commercial vendor.

Surgical ligation of the stifle ligament and medial meniscectomy in wildtype and C5 knock out mice was performed (Stanton, H. et al., Nature, 434:649-52 (2005); Clements, K. M. et al., Arthritis Rheum. 48:3452-63 (2003)). Briefly, the mice were anesthetized, and the left stifle ligament exposed and severed with a scalpel, the medial meniscus removed, and skin glue used to close the wound. One week or 16 weeks post-surgery, mice are sacrificed and both hind stifle joints harvested, fixed in paraformaldehyde, decalcified, and sections stained with H&E and/or toluidine blue. Tissues images of wildtype mice one week and 16 weeks post surgery are shown in FIGS. 3A and 3B, respectively. Tissue images of mice 16 weeks after surgery in wildtype and C5-deficient mice are shown in FIGS. 4A-4B, respectively. The cartilage surface from the operated joints in wildtype mice demonstrated extensive proteoglycan and cartilage loss (FIG. 4A, four larger arrows) and osteophyte formation (two smaller arrows at edge of image). In contrast, the cartilage and proteoglycan were preserved in C5 deficient mice and there was no significant osteophyte development (FIG. 4B).

### Example 2

### Generation of Animals Genetically Deficient for Complement Components

Mice genetically deficient for factors representing various catalytic arms of the complement pathway (C3, C4, C5, C6, MBL, factor B), anaphylatoxin receptors (C5 receptor), and natural inhibitors (CD59) of the complement system are generated and examined to determine the roles of these factors in the development of osteoarthritis. Each of the mouse strains listed in the table below is tested for an osteoarthritis phenotype following surgical joint injury. Transgenic mice overexpressing a natural complement inhibitor (Crry) are studied to determine if they are protected against development of osteoarthritis.

| Pathway/Role in | | |
|---|---|---|
| Complement System | Gene | Source of mouse strain |
| Central common pathway | C5-deficient | Jackson # 000461 |
| | C3-deficient | Jackson # 003641 |
| Classical pathway | FcRy-deficient | Taconic Farms # |
| | | 000583 |
| | Rag1-deficient | Jackson # 002216 |
| | Igh-6-deficient | Jackson # 002249 |
| | (heavy chain of IgM) | |
| | C4-deficient | V. M. Holers |
| | | (Univ. Colorado) |
| Alternative pathway | Factor B-deficient | V. M. Holers |
| | | (Univ. Colorado) |
| Mannose-binding lectin | Mbl1; Mbl2-deficient | Jackson # 006122 |
| (MBL) pathway | | |
| Anaphylatoxin effector | C5a Receptor - | Jackson # 005616 |
| | deficient | |
| MAC effector pathway | C6-deficient | T. Wyss-Coray |
| | | (Stanford) |
| Complement regulatory | CD59a-deficient | T. Wyss-Coray |
| proteins | | (Stanford) |
| | Crry transgenic | V. M. Holers |
| | | (Univ. Colorado} |

### Example 3

### Quantitative Assay on Degree of Osteoarthritis in Wildtype and C5-Deficient Mice

Groups of C5-deficient (n=5; C57BL/10 (B10) background) and wildtype B10 control (WT, n=5) mice were surgically-induced to develop degenerative osteoarthritis, as described in Example 1, After 4 months, the mice were sacrificed, the stifle joint was harvested, fixed in paraformaldehyde, decalcified, and sections stained with toluidine blue. The stained stifle joint sections were visualized and scored for the degree of posttraumatic osteoarthritis by a blinded examiner, using a previously described scoring system (Bendele A. M., J. Musculoskelet. Neuronal Interact., 2:501-3 (2002)). In brief, a composite scoring system was used to quantitate the degree of degenerative osteoarthritis in histology sections derived from the surgically-induced mice. The degenerative arthritis or “OA score” was based depth of proteoglycan loss in each joint quadrant (femoral-media, femoral-lateral, tibial-medial, tibial-lateral). The depth of proteoglycan loss is then multiplied by the length within that quadrant over which that degree of proteoglycan loss is observed. The “OA score” is then calculated as the sum of values for all four quadrants. The ligated joints were compared to unoperated joints.

Results are shown in FIG. 5. FIG. 5A shows that C5-deficient mice developed statistically less severe degenerative arthritis in their surgically injured left (L) stifle joint as compared to wildtype (WT) controls (p=0.04). The unoperated right (R) stifle joints exhibited no significant arthritis and no statistical differences in scores between C5 KO and WT mice. Blinded scoring of toluidine blue stained stifle joint sections from C5 deficient (n=5) and wildtype (n=5) mice surgically induced for OA demonstrated statistically significant protection against development of degenerative arthritis in the C5-deficient mice (p<0.03 by t-test). In FIGS. 5B and 5C, results from a timecourse experiment are presented, in which C5-deficiency protected against the progressive development of OA and gait dysfunction. In FIG. 5B, histological analysis of the OA phenotype was performed at several time points after surgical destabilization of the right stifle joint. Photomicrographs of representative toluidine blue-stained joint sections at several time points after surgery show progressive proteoglycan and cartilage loss in C5+, but not C5−, mouse joints. In FIG. 5C, the degree of proteoglycan and cartilage loss was quantitated. C5+ mice developed significant progression of the OA phenotype as compared to C5− mice (*P≦0.05 by Student's t-test), and corresponding mice that had not undergone surgical destabilization (C5+ non-operated and C5− non-operated). Error bars represent SEM. The results suggest that protection against complement-mediated cartilage degeneration results in protection of articular cartilage on histologic analysis.

### Example 4

### Gait Analysis of C5-Deficient Mice and Wildtype Mice

Gait analysis was performed using the Noldus CatWalk Gait Analysis System, shown in FIG. 6A, developed to characterize neurologic deficits in mice (Gabriel A. F. et al., J. Neurosci. Methods, 163(1):9-16 (2007)). In this system mice walk on a glass catwalk, a camera captures images and quantitates pressure exhibited by each paw, and a computer analyzes the “stride pattern activity”.

C5-deficient (n=5) and wildtype (n=5) mice were placed individually on the Catwalk System 16 weeks following surgical induction of degenerative arthritis (Example 1). Each mouse walked the cat walk three times, and the CatWalk system analyzed the stride patterns. Data acquisition started with the rodent traversing over the walkway. As seen in FIGS. 6A-6C, light that entered the walkway's glass floor was internally reflected, except for those places where the animal's body makes contact with the floor. At those places, light escapes, with each contact point resulting in a separated illuminated area. A video camera positioned underneath the glass floor captured the illuminated areas and sent the video image to a computer. The user assigned labels to the illuminated areas, and the software was programmed to analyze the gait data. The width and length of individual footprints, distance between two successive placements of a paw (stride length), and pressure exerted on the floor are examples of the parameters calculated. Static gait parameters, the CatWalk system may be used to calculate a variety of time-related parameters, such as the swing and stance duration and swing speed.

Normal mice utilize stride pattern “Ab” for 70-80% of strides, defined as the sequence of paw strides being RF-RH-LF-LH (right front—right hind—left front—left hind). Four months following surgical induction for OA, C5 deficient mice exhibited the normal stride pattern “Ab” while wildtype mice (with severe OA) utilize this pattern significantly less. (P=0.03 by T test). FIG. 7A shows the frequency of usage of each gait pattern for each of five wildtype mice (circles) and five C5-knockout mice (boxes). A red circle for each mouse is present in each of the four categories to show the frequency of gait pattern in percentage terms. The results shown in FIG. 7A demonstrate that following surgical joint injury, C5-deficient mice maintain a normal gait while wildtype mice exhibit abnormal gait.

C5-deficient mice that were resistant to development of posttraumatic OA maintained the normal “Ab” stride pattern, while wildtype mice that developed severe degenerative arthritis exhibited statistically less frequent use of the “Ab” stride pattern (***p=0.03 by T Test). Healthy mice use the stride pattern “Ab” in 70-80% of their gait, and stride patterns “Ca”, “Cb”, and “Ab” in <20% of gait.

FIG. 7B, shows the gait analysis results from the mice described in FIGS. 5B and 5C. Gait was analyzed at serial time points after surgical destabilization in C5+ and C5− mice, and also in corresponding mice that had not undergone surgical destabilization (C5+ non-operated and C5− non-operated). Compared to mice in the other groups, osteoarthritic C5+ mice used the Ab stride pattern progressively less (*P≦0.05 by Student's t-test). At week 8, n=6 for C5+ operated, n=6 for C5− operated; and at week 12, n=3 for C5+ operated and n=3 for C5− operated.

The results suggest that protection against complement-mediated cartilage degeneration results in improvement and functional normalization of gait.

### Example 5

### Mice Deficient in Antibody Receptors, Mannose-Binding Lectin (MBL-1 and MBL-2) or C5a Receptor 1 (C5aR1) are Not Resistant to Osteoarthritis Pathogenesis

Mice genetically deficient for FcγRIIb (n=5) or FcγRIII (n=5), along with background-matched controls (n=5 for each experiment) were subject to surgical induction of degenerative arthritis in the left (L) stifle joint. Blinded scoring of toluidine blue-stained histologic sections demonstrated development of degenerative arthritis in the operated stifle joints. The results are shown in FIG. 8. The results suggest that the FcγRIIb and FcγRIII are not critical to development of the osteoarthritis phenotype in this model.

Mice genetically deficient for MBL-1 and MBL-2 (n=5), along with background-matched controls (n=5 for each experiment) were subject to surgical induction of degenerative arthritis in the left (L) stifle joint. Blinded scoring of toluidine blue-stained histologic sections demonstrated development of degenerative arthritis in the operated stifle joints. The results are shown in FIG. 8. The results suggest that the MBL-1 and MBL-2, and thus the mannose-binding lectin pathway, are not critical to development of the osteoarthritis phenotype in this model.

Mice genetically deficient for C5aR1 (n=5), along with background-matched controls (n=5 for each experiment) were subject to surgical induction of degenerative arthritis in the left (L) stifle joint. Blinded scoring of toluidine blue-stained histologic sections demonstrated development of degenerative arthritis in the operated stifle joints. The results are shown in FIG. 8. The results suggest that the C5aR1, and thus the anaphylatoxin effector pathway, is not critical to development of the osteoarthritis phenotype in this model.

### Example 6

### Immunohistochemistry and ELISA Studies on Human Osteoarthritis Joint Samples

Immunohistochemical staining was performed on remnant osteoarthritis cartilage obtained from a human patient at the time of knee arthroplasty. Cartilage samples were prepared and stained with antibodies specific for C3c (central complement pathway), MAC (C5b-9) (membrane attack complex effector), and C5aR (chemoattraction/inflammation effector). Exemplary antibodies that target these pathways are shown in the table below. Results are shown in FIGS. 9-10. The results demonstrate the presence and suggest the activation of the central complement component C3 resulting in the formation of the MAC in human osteoarthritis cartilage. The results suggest that complement activation contributes to the breakdown of cartilage and the death of chondrocytes in human osteoarthritis.

Exemplary antibodies for immunohistochemistry
on mouse joint sections

| Antibody | Target/Pathway | Source | Specificity |
|---|---|---|---|
| Anti-C3c | Central | Abcam # | rabbit anti-mouse C3c, |
| | complement | ab4212 | recognizes C3c, C3b, and C3 |
| | pathway | | |
| Anti-MAC | Effector | Abcam # | Rabbit anti-mouse C5b-9 |
| (C5b-9) | | ab55811 | |
| Anti-C5a | Anaphalatoxin | Serotec | Rat anti-mouse C5a |
| receptor | effector | MCA2456 | Receptor |

An enzyme-linked immunosorbent assay (ELISA) to measure MAC (C5b-9) was performed on synovial fluid derived from subjects with osteoarthritis, rheumatoid arthritis, gout, calcium pyrophosphate crystal disease. ELISA to measure MAC was also performed on human serum. Results are presented in FIG. 11A, and demonstrate a range of levels of MAC in human osteoarthritis synovial fluid, with approximately ⅔ of osteoarthritis patients exhibiting elevated levels suggesting activation of the complement system, and ⅓ of patients exhibiting low levels similar to those detected in synovial fluid derived from healthy individuals. More than ⅔ of osteoarthritis patients also exhibited elevated levels of complement C3a, measured in ng/mL, in their synovial fluid samples as compared to synovial fluid samples from healthy individuals. These results demonstrate that a subset of patients with osteoarthritis have elevated levels of the MAC (C5b-9; terminal complement complex) and complement C3a in the synovial fluid of affected joints, suggesting ongoing activation of the complement cascade in these osteoarthritis patients.

### Example 7

### In Vitro Complement Activation Studies

Human serum reagents as complement sources were obtained as summarized in the table below. Factor B-depleted sera lack the alternative pathway factor and C1q-depleted sera lacks the classical pathway factor.

Exemplary reagents for in vitro complement activation and reconstitution experiments

| Reagent | Source | Target/Pathway | Comments |
|---|---|---|---|
| Normal human serum | Quidel #A113 | For in vitro complement | Diluted to 10% in PBS for in vitro |
| complement | | activation assays | complement activation assays. |
| Factor B-depleted | Quidel #A506 | Lacking alternative | Diluted to 10% in PBS for in vitro |
| human sera | | pathway factor | complement activation assays. |
| C1q-depleted human | Quidel #A509 | Lacking classical pathway | Diluted to 10% in PBS for in vitro |
| sera | | factor | complement activation assays. |

Cartilage component proteins, itemized in a table below, were used to activate the complement system in the serum reagents, and MAC (C5b-9) was analyzed quantitatively by ELISA as a measure of complement activation. In addition to recombinant and purified cartilage components, pulverized human osteoarthritis cartilage, pulverized non-osteoarthritis cartilage, and pulverized human osteoarthritis synovium were also tested for their ability to activate the complement system as measured by the amount of MAC produced. Sepharose (Sepharose 4B agarose beads) and zymosan are well established to potently activate the complement system, and were used as positive controls in these experiments Phosphate buffered saline (PBS) was used as a negative control in these experiments.

Exemplary cartilage components

| Cartilage | | |
|---|---|---|
| component | Source | Comments |
| Fibromodulin | Novus #H00002331- | Recombinant human protein |
| | P01 | with GST. |
| Aggrecan | Sigma #A1960 | Purified from bovine articular |
| | | cartilage. |
| Type II | Sigma #C1188 | Purified from bovine tracheal |
| collagen | | cartilage. |
| Matrilin-3 | R&D Systems #3017- | Recombinant human protein. |
| | MN | |
| CLIP | Novus #H00008483- | Recombinant human protein |
| | Q01 | with GST. |
| COMP | R&D Systems #3134- | Recombinant human protein, |
| | CP | polyhistidine tag. |

The results in FIG. 12 demonstrate that pulverized osteoarthritis cartilage, but not pulverized osteoarthritis synovium tissue, activates the complement system to result in the production of MAC. Addition of ethylene diamine tetraacetic acid (EDTA), a chemical that chelates divalent and trivalent cations which are needed for activation of the complement system, inhibited activation of the complement system by pulverized osteoarthritis cartilage. Agarose beads (Sepharose 4B), which are well established to activate the complement system, represents a positive control and potentially activated the complement system to result in production of MAC. In contrast, phosphate buffered saline (PBS) did not activate the complement system and did not result in production of MAC. The results in FIG. 13 demonstrate that in contrast to pulverized osteoarthritis cartilage which activated the complement system to produce MAC, non-osteoarthritis cartilage (KI cart) did not activate the complement system. The results in FIG. 14A demonstrate that the cartilage component fibromodulin activates the complement system to result in the production of MAC, while the cartilage components collagen type II and matrilin-3 did not activate the complement system. FIG. 14B demonstrates the elevation of fibromodulin in synovial fluid samples derived from osteoarthritis patients as compared to healthy individuals, and in FIG. 14C that elevated levels of fibromodulin are associated with increased levels of C3a in the synovial fluid. These result suggests that the cartilage component fibromodulin and potentially other cartilage components released in the injury or breakdown of cartilage active and/or perpetuate the activation of the complement system, thereby causing further injury to the cartilage matrix and/or chondrocytes which results in the development or progression of osteoarthritis.

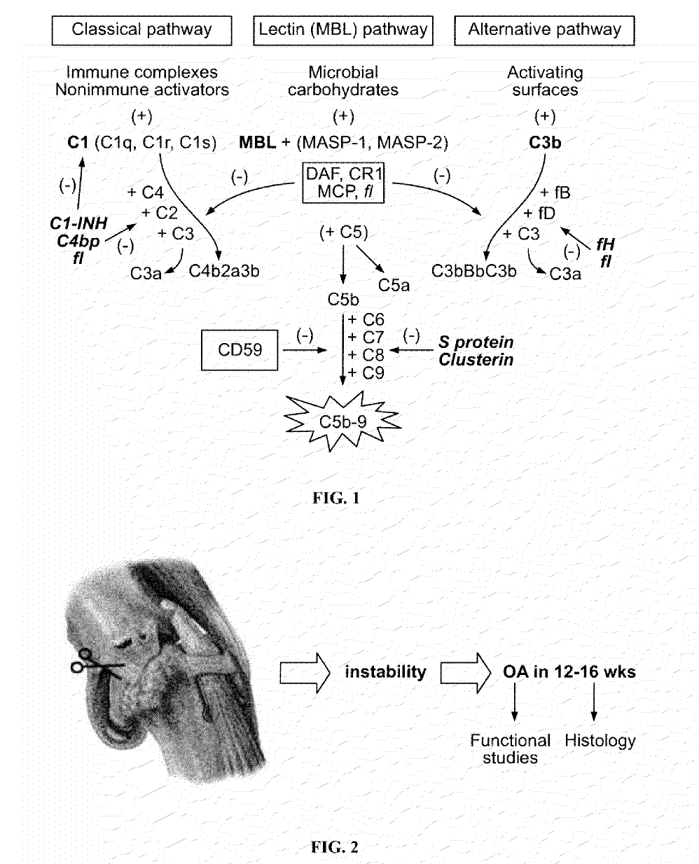

The results in FIG. 15 demonstrate that pulverized osteoarthritis cartilage does not active the complement system in serum depleted of factor B. The results in FIG. 16 demonstrate that anti-factor B antibodies (anti-Bb and anti-BB) also inhibited activation of the complement system by pulverized osteoarthritis cartilage. These results demonstrate that factor B and thus the alternative complement pathway (FIG. 1) plays an important role in the activation of the complement system by components present in osteoarthritis cartilage. These results suggest that the alternative complement pathway contributes to complement-mediated damage of the cartilage matrix and/or chondrocytes in osteoarthritis.

### Example 8

### Complement Activation and Inflammatory Response

Time course experiments are performed in mice surgically induced for OA to assess whether activation of complement and generation of MAC precede elevations in inflammatory cytokines and cartilage-degrading proteolytic activity, or vice versa. Sixteen C57BL/10 (B10) mice are surgically induced to develop OA (Example 1), with two mice sacrificed for analysis at each time point (weeks 1, 2, 3, 4, 6, 8, 12 and 16). To determine whether arthritic degeneration is initiated by complement activation, which then leads to inflammatory cytokine release and cartilage breakdown, or vice versa, stifle joints are harvested and characterized by immunohistochemistry using antibodies, listed in the table below, and specific for: (i) C3c and MAC as readouts for complement activity, (ii) IL-1 and TNF as representative inflammatory cytokines, and (iii) the aggrecan C-terminal neoepitope generated by the aggrecanase ADAMTS-5, which is upregulated by IL-1. The tissue sections are stained for hemoglobin (Okajima's stain) and hemosiderin (Prussian blue stain) to assess resolution of hemearthrosis.

Exemplary antibodies used for immunohistochemistry time course experiments

| Antibody | Target/Pathway | Source | Specificity |
|---|---|---|---|
| Anti-C3c | Central complement | Abcam # ab4212 | rabbit anti-mouse C3c, |
| | pathway | | recognizes C3c, C3b, C3 |
| Anti-MAC (C5b-9) | Effector | Abcam # ab55811 | Rabbit anti-mouse C5b-9 |
| Anti-IL-1α | Inflammatory cytokine | R&D #AF-400-NA | Goat-anti-mouse |
| Anti-TNF | Inflammatory cytokine | R&D #AF-410-NA | Goat-anti-mouse |
| Anti-CNITEGE | Aggrecan epitope following | Rabbit polyclonal to | |
| | aggrecanase cleavage | CNITEGE | |

Immunohistochemical and ELISA analyses demonstrate complement activation through the alternative pathway in a significant fraction of OA cartilage and synovial fluid samples. Immunohistochemical staining, immunoblotting and ELISA analysis of membrane-bound and soluble inhibitors, especially the alternative pathway regulator factor H, is performed to establish that the levels or activity of factor H and other inhibitors are altered. Time course experiments show that complement activation and generation of MAC occurs prior to elevations in IL-1, TNF and generation of aggrecan-cleaved neoepitopes, supporting the “Complement-Driven Model” illustrated in FIG. 17.

### Example 9

### Inhibition of Molecules in the Alternative Complement Pathway Eliminates the OA Cartilage-Induced Activation of Complement

Inhibition of factor B eliminates the osteoarthritis cartilage-induced activation of complement. Normal human serum was preincubated with murine monoclonal antibody to factor B (anti-Bb and anti-B) for one hour at 4° C., Pulverized osteoarthritis cartilage (OA cart), PBS, Sepharose 4b (seph 4b), or aggregated human IgG (AHG) was then added, followed by quantitation of MAC by ELISA at various time points. Results from triplicate wells are presented, and error bars represent standard error of the mean (SEM). In the absence of anti-factor B antibody, OA cartilage, sepharose 4b, and aggregated human IgG activate complement. Activation by sepharose 4b, which occurs via the alternative pathway, is eliminated by preincubation with anti-factor B antibody (FIG. 16). The same is true for OA cartilage (FIG. 16). Anti-factor B antibody does not block activation by human IgG, which occurs via the classical pathway. In this example, the low level of MAC produced is likely a consequence of tickover, which occurs downstream of where factor B is located in the pathway. The result suggests that inhibition of the alternative complement pathway with anti-factor B antibody or antagonists of other components of the alternative pathway could inhibit osteoarthritis cartilage component-mediated activation of complement and thereby provide benefit in preventing and treating osteoarthritis.

### Example 10

### Treatment with Recombinant Complement Inhibitors to Prevent the Development of Degenerative Arthritis Following Surgical Joint Injury in Mice

Groups of 5 mice per experimental arm are treated with a recombinant complement inhibitor or with a control antibody. Two treatment regimens are used. In the first, continuous treatment is applied for 16 weeks following surgical joint injury through the time of sacrifice. In the second, treatment is applied for a four week period starting immediately following surgical joint injury, after which mice are left untreated for three additional months. The mice in both treatment groups are sacrificed 16 weeks after surgical joint injury, and the joint is removed for histologic analysis. Mice treated with the antibody that inhibits the complement component do not develop osteoarthritis, whereas the mice treated with a control antibody show evidence of osteoarthritis. Exemplary complement inhibitors are outlined in the table below, and include a monoclonal antibody against complement component C5, a monoclonal antibody against factor B, a CR2-Crry recombinant fusion protein, and a CR2-Factor H recombinant fusion protein.

TABLE 7
Recombinant complement inhibitors (and control treatments)
to be tested in the murine model of posttraumatic OA.

| Inhibitor | Mechanism | Dose and route | Source and reference |
|---|---|---|---|
| Anti-C5 Ab | Blocks C5 | 750 ug/IP 2× per wk | V. M. Holers |
| | | | (Banda et al, 2002; Wang et al, 1995) |
| Anti-factor B Ab | Blocks factor B | 1000 ug/IP 3× per wk | V. M. Holers |
| CR2-Crry | Inhibits C3 | 0.25 mg IV once per wk | V. M. Holers |
| | convertase | | (Song et al, 2007; Atkinson et al, 2005) |
| CR2-Factor H | Inhibits C3 | 0.25 mg IV once per wk | V. M. Holers |
| | convertase | | |
| Istoype ctrl. Ab | Control | 750 ug/IP 2× per wk | Sigma |

### Example 11

### Administration of a Complement Inhibitor to a Patient at Risk for Developing Osteoarthritis

A patient who has a family history of degenerative osteoarthritis is evaluated by a medical doctor for the risk of developing osteoarthritis. This evaluation can include a medical history and/or physical examination of the joints, x-ray or magnetic resonance imaging of joints, blood testing, and/or genetic testing. Genetic testing is performed by analysis of single-nucleotide polymorphisms (SNPs) in the patient's genome using microarrays (such as the Illumina or Affymetrix technologies) or by DNA sequencing. If the patient is determined to be at significant risk for the development of osteoarthritis, a complement inhibitor is administered in a therapeutic dosage form such that it is effective to inhibit the complement pathway in the joints that exhibit symptoms of osteoarthritis. An exemplary complement inhibitor that may be used is a human monoclonal antibody against complement component C5.

The patient is treated with the anti-C5 antibody Soliris® (eculizumab, available from Alexion), which is administered in a dose according to label instructions, where Soliris® is administered as an IV infusion by a health care provider. Alternatively, the anti-C5 antibody Soliris® is injected directly into the joint developing or at risk for developing osteoarthritis. The patient does not develop osteoarthritis over the monitoring period.

### Example 12

### Administration of a Complement Inhibitor to a Patient in Need of Osteoarthritis Treatment

To a patient who has been diagnosed with osteoarthritis, a complement inhibitor is administered in a therapeutic dosage form such that it is effective to inhibit the complement pathway in the joints that exhibit signs of osteoarthritis. An exemplary complement inhibitor that may be used for treatment is the anti-C5 antibody.

A 62 year old man has a history of osteoarthritis in his right knee. This patient has been treated with pain-management medications. The patient is treated with the anti-C5 antibody Soliris® (eculizumab, available from Alexion), which is administered in a dose according to label instructions, where Soliris® is administered as an IV infusion by a health care provider. For the first four weeks, 600 mg each week is administered. Thereafter, additional doses are administered until a satisfactory clinical or biomarker endpoint is reached. Alternatively, the anti-C5 antibody Soliris® is injected directly into the joint affected by osteoarthritis. For this patient, a satisfactory clinical endpoint is slowing of the progression of osteoarthritis and possibly improvement in joint pain and function. Treatment may be repeated as often as needed if the osteoarthritis symptoms or biomarkers return.

### Example 13

### Administration of a Complement Inhibitor to Prevent Development of Osteoarthritis

To a patient in which there is a desire to prevent development of osteoarthritis, a complement inhibitor is administered in a therapeutic dosage form such that it is effective to inhibit the complement pathway in the joint(s) at risk for the development or progression of osteoarthritis. An exemplary complement inhibitor that may be used for treatment is the anti-C5 antibody.

A 41 year old woman who jogs frequently is asymptomatic but concerned about developing osteoarthritis based on her family history. Magnetic resonance imaging demonstrates mild abnormalities of the cartilage, suggesting the presence of early-stages of cartilage breakdown. The patient is treated with the anti-C5 antibody Soliris® (eculizumab, available from Alexion), which is administered in a dose according to label instructions. Specifically, Soliris® is administered as an IV infusion or by an intra-articular injection by a health care provider. The dosage is adjusted such that the amount and frequency of administration prevent development of osteoarthritis, as measured by periodic radiographic imaging of the knee joints in the patient or by measurement of other biomarkers.

### Example 14

### Administration of a Complement Inhibitor to Prevent Development of Osteoarthritis Following Joint Trauma

To a patient in which there is a desire to prevent development of osteoarthritis, a complement inhibitor is administered in a therapeutic dosage form such that it is effective to inhibit the complement pathway in the joint(s) at risk for the development of osteoarthritis. An exemplary complement inhibitor that may be used for treatment is the anti-C5 antibody.

A 30 year old man who plays recreational soccer twists his knee in a soccer match. Following the injury the knee swells and is painful, and a magnetic resonance image demonstrates a torn anterior cruciate ligament. The patient is treated with the anti-C5 antibody Soliris® (eculizumab, available from Alexion), which is administered in a dose according to label instructions. Specifically, Soliris® is administered as an IV infusion or by an intra-articular injection by a health care provider. The dosage is adjusted such that the amount and frequency of administration prevent development of osteoarthritis, as measured by periodic radiographic imaging of the knee joints in the patient or by measurement of other biomarkers.

### Example 15

### Administration of a Complement Inhibitor to Prevent Development of Osteoarthritis Following Joint Injury

To a patient who has or is sustaining joint injury in which there is a desire to prevent development of osteoarthritis, a complement inhibitor is administered in a therapeutic dosage form such that it is effective to inhibit the complement pathway in the joint(s) at risk for the development of osteoarthritis. Exemplary joint injuries include physically traumatic joint injuries, fractures involving joints, joints with cartilage damage, joints with ligament damage, hemearthrosis, surgical instrumentation of joints, microbial infection of joints, gout and pseudogout attacks in joints, autoimmune diseases involving joints (including rheumatoid arthritis, psoriatic arthritis, ankylosing spondylitis, Reiter's arthritis, reactive arthritis, juvenile rheumatoid arthritis, systemic lupus erythematosus), and other joint insults that may result in the development of osteoarthritis years to decades later.

An exemplary complement inhibitor that may be used for treatment is the anti-C5 antibody. The patient is treated with the anti-C5 antibody Soliris® (eculizumab, available from Alexion), which is administered in a dose according to label instructions. Specifically, Soliris® is administered as an IV infusion by a health care provider. Alternatively, Soliris® is administered intra-articularly into the involved joints. The dosage is adjusted such that the amount and frequency of administration prevent development of osteoarthritis, as measured by periodic monitoring of the patient. Long-term therapy may be necessary in certain patients, but in other patients it may be possible in the post-traumatic setting to administer a brief treatment period of one dose to 12 months of therapy to prevent initial activation of the complement cascade and thereby circumvent the inflammatory cycle that leads to OA. Use of a brief and defined treatment period reduces the risk of infectious and other complications that arise from long-term use of complement inhibitors.

### Example 16

### Method of Identifying Patients at Risk for Developing Osteoarthritis or Experiencing Progression of Osteoarthritis Who Would Benefit from Treatment with a Complement Inhibitor

Clinical history, clinical examination, blood biomarker analysis synovial fluid analysis, joint imaging studies, and/or genetic analyses may all facilitate the identification of individuals at risk for developing osteoarthritis or experiencing progression of osteoarthritis. Osteoarthritis is diagnosed by first considering and “ruling out” other possible disorders (UpToDate, 16.1 (2008), www.uptodate.com).

Clinical history and examination criteria for identifying individuals at increased risk for developing osteoarthritis, or experiencing progression of osteoarthriits, include joint pain, increasing age, morning stiffness <30 minutes, crepitus on active motion, bony tenderness, and bony enlargement. Evidence of osteoarthritis in other joint, such as the distal interphalangeal joints of the fingers or first carpal metacarpal joint of the thumb, may identify patients at increased risk for developing or experiencing progression of osteoarthritis in other joints such as the knees and hips. Nevertheless, many individuals who will develop osteoarthritis do not have significant joint pain, stiffness or other symptoms during the early stages of disease initiation and progression.

Enzyme-linked immunosorbent assays (ELISA) or other singleplex or multiplex (proteomic) immunoassays may be used to determine the presence of complement molecules, cartilage breakdown products, cytokines, and/or other biomarkers in the blood and/or synovial fluid of patients being evaluated. An individual patient's protein biomarker profile, based on detection of complement components, cartilage breakdown products, cytokines, and/or other biomarkers, is then compared to OA and control genetic profiles to determine the risk of that patient for developing OA.

Imaging techniques, such as X-ray, MRI, CT, microCT, or ultrasound, may demonstrate cartilage breakdown and other radiographic features (subchondral cysts, subchondral sclerosis, and osteophyte formation) that facilitate identification of individuals at risk for developing or more likely to experience progression of osteoarthritis. These imaging techniques may also demonstrate mild cartilage abnormalities that suggest the future development of osteoarthritis.

Genetic risk for the development of OA can be assessed by isolating genomic DNA from the patient's blood, and then performing single nucleotide polymorphism (SNP) analysis by microarray analysis (using technologies from Illumina or Affymetrix) or DNA sequencing. An individual patient's genetic profile (based on SNP analysis or DNA sequencing) is then compared to OA and control genetic profiles to determine the risk of that patient for developing OA.

In one embodiment, patients with joint pain or joint injury or who are identified to possess genetic risk for the development of osteoarthritis are further screened with clinical history, physical examination, blood tests, genetic testing, and radiographic imaging studies. An “osteoarthritis risk algorithm” is then used to integrate the results from clinical history, clinical examination, blood biomarkers, synovial fluid biomarkers, joint imaging studies, and/or genetic testing to identify individuals at increased risk for developing or experiencing progression of osteoarthritis. Patients with at risk for developing osteoarthritis or at increased risk for progression of osteoarthritis are then treated with a complement inhibitor.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

It is claimed:

1. A method of treating a subject at risk of developing or currently having osteoarthritis, comprising
administering to the subject a complement inhibitor compound in a therapeutically effective amount

2. The method according to claim 1, wherein said administering is to a subject at risk for developing post-injury osteoarthritis.

3. The method according to claim 1, wherein said administering is to a subject with a genetic tendency to osteoarthritis.

4. The method according to claim 1, wherein said compound is an inhibitor of a molecule in the alternative complement pathway.

5. The method according to claim 1, wherein said compound is an inhibitor of a molecule in the classical complement pathway.

6. The method according to claim 1, wherein said compound is an inhibitor of a molecule in both the alternative and classical complement pathways.

7. The method according to claim 1, wherein said compound is an inhibitor of a molecule selected from C5, C5a, and C5b.

8. The method according to claim 1, wherein said compound is an inhibitor of the membrane attack complex.

9. The method according to claim 1, wherein the compound is a polypeptide.

10. The method according to claim 9, wherein the polypeptide is a CR2-Factor H fusion protein.

11. The method according to claim 9, wherein the polypeptide is a CR2-Crry fusion protein.

12. The method according to claim 1, wherein the compound is an antibody or functional fragment thereof.

13. The method according to claim 12, wherein the compound is an antibody specific for complement component C5.

14. The method according to claim 1, wherein the compound is a small molecule.

15. The method according to claim 1, wherein said compound prevents cleavage of a complement molecule to its fragments.

16. The method according to claim 15, wherein the compound prevents cleavage of C2, C3, C4, or C5.

17. The method according to claim 1, further comprising administering to the subject an anti-inflammatory agent, an analgesic, or a steroid.

18. The method according to claim 1, further comprising treating the patient with a physical therapy exercise.

19. A method of preventing, delaying, or reducing the probability of development of osteoarthritis in a human subject, comprising:
administering to the subject a complement inhibitor compound in a therapeutically effective amount.

20. A method of slowing or inhibiting the progression of osteoarthritis in a subject at risk of developing osteoarthritis or currently having osteoarthritis, comprising:
administering to the subject a complement inhibitor compound or a functional equivalent thereof in a prophylactically or therapeutically effective amount.
